(19) United States
(12) Patent Application Publication
(10) Pub. No.: US 2024/0115838 A1
(43) Pub. Date: Apr. 11, 2024

(54) BALLOON CATHETER

(71) Applicant: THE CLEVELAND CLINIC FOUNDATION, Cleveland, OH (US)

(72) Inventor: Karunakaravel Karuppasamy, Cleveland, OH (US)

(21) Appl. No.: 18/482,074

(22) Filed: Oct. 6, 2023

Related U.S. Application Data
(60) Provisional application No. 63/414,029, filed on Oct. 7, 2022.

(51) Int. Cl. A61M 25/10; A61M 25/00

(52) U.S. Cl. CPC A61M 25/1002; A61M 25/003; A61M 25/104; A61M 2025/0037; A61M 2025/1061

(57) ABSTRACT

A balloon catheter includes a catheter having longitudinally opposite proximal and distal catheter end portions. The catheter has a catheter inflation opening. An expandable balloon is positioned on at least the distal catheter end portion. The balloon defines at least one balloon opening. A first balloon catheter lumen extends substantially longitudinally from the proximal catheter end portion toward the distal catheter end portion. A second balloon catheter lumen extends substantially longitudinally from the proximal catheter end portion to at least the at least one balloon opening. An inflation lumen extends substantially longitudinally from the proximal catheter end portion to the catheter inflation opening. The catheter inflation opening provides fluid communication between the inflation lumen and an inflatable volume of the expandable balloon.

### RELATED APPLICATION

This application claims priority from U.S. Provisional Application No. 63/414,029, filed 7 Oct. 2022, the subject matter of which is incorporated herein by reference in its entirety.

### TECHNICAL FIELD

This disclosure relates to one or more balloon catheters, one or more methods for manipulating a patient lumen (e.g., using a balloon catheter), and one or more methods for constructing a balloon catheter.

### BACKGROUND

Certain medical procedures include advancing a balloon catheter over a guidewire to a target site within a patient lumen. The target site may be a portion of the patient lumen in which an undesirable stenosis and/or occlusion is present. Once in position, a balloon of the balloon catheter is inflated to dilate the target site, and thereby may at least partially help alleviate the stenosis and/or occlusion. The balloon catheter may also carry an expandable stent that is deployed at the target site as the balloon is inflated. The deployed stent may at least partially help prevent the stenosis and/or occlusion from reoccurring for a period of time. The balloon may also be used for localized application of drugs to reduce restenosis. Known balloon catheters used in the above manner may be inadequate in certain use environments, such as, for example, when the target site is adjacent to a patient lumen branch that extends from the patient lumen.

### SUMMARY

In an aspect, alone or in combination with any other aspect, A balloon catheter comprises a catheter having longitudinally opposite proximal and distal catheter end portions. The catheter has a catheter inflation opening. An expandable balloon is positioned on at least the distal catheter end portion. The balloon defines at least one balloon opening. A first balloon catheter lumen extends substantially longitudinally from the proximal catheter end portion toward the distal catheter end portion. A second balloon catheter lumen extends substantially longitudinally from the proximal catheter end portion to at least the at least one balloon opening. An inflation lumen extends substantially longitudinally from the proximal catheter end portion to the catheter inflation opening. The catheter inflation opening provides fluid communication between the inflation lumen and an inflatable volume of the expandable balloon.

In an aspect, alone or in combination with any other aspect, a method for manipulating a patient lumen comprises providing a balloon catheter. A first guidewire is inserted into a target patient tissue site in a patient lumen with a first guidewire distal end of the first guidewire being positioned in the patient lumen. A second guidewire is inserted into the target patient tissue site with a second guidewire distal end of the second guidewire being positioned in a patient lumen branch that extends from the patient lumen. A first guidewire proximal end of the first guidewire is directed through the first balloon catheter lumen. A second guidewire proximal end of the second guidewire is directed through the second balloon catheter lumen. The balloon catheter is directed to the target patient tissue site along the first and second guidewires. The balloon catheter is positioned at the target patient tissue site with at least a portion of the catheter and at least a portion of the expandable balloon being positioned in the patient lumen, and with the balloon opening directed toward the patient lumen branch. With the balloon catheter positioned at the target patient tissue site, the balloon is inflated to dilate the patient lumen. The balloon is deflated after the patient lumen has achieved a predetermined amount of dilation.

In an aspect, alone or in combination with any other aspect, a balloon catheter comprises a catheter having longitudinally opposite proximal and distal catheter end portions. The distal catheter end portion has a distal catheter sidewall opening. At least one expandable balloon is positioned on at least the distal catheter end portion. The at least one balloon defines a balloon opening aligned with the distal catheter sidewall opening. A first balloon catheter lumen extends substantially longitudinally from the proximal catheter end portion to the distal catheter tip. A second balloon catheter lumen extends substantially longitudinally from the proximal catheter end portion to at least the balloon opening. An inflation lumen extends substantially longitudinally from the proximal catheter end portion to a catheter inflation opening. The catheter inflation opening provides fluid communication between the inflation lumen and an inflatable volume of the expandable balloon.

In an aspect, alone or in combination with any other aspect, a balloon catheter comprises a catheter having longitudinally opposite proximal and distal catheter end portions. The distal catheter end portion has a distal catheter sidewall opening. An expandable balloon is positioned on at least the distal catheter end portion. The balloon defines a balloon opening aligned with the distal catheter sidewall opening. The balloon has a balloon inflation opening in fluid communication with an inflatable volume of the balloon. The balloon inflation opening is aligned and in fluid communication with a catheter inflation opening of the catheter. A first balloon catheter lumen extends substantially longitudinally from the proximal catheter end portion to the distal catheter tip. A second balloon catheter lumen extends substantially longitudinally from the proximal catheter end portion to at least the balloon opening. An inflation lumen extends substantially longitudinally from the proximal catheter end portion to the balloon inflation opening such that inflation fluid is selectably directed into the inflatable volume of the balloon through both the catheter inflation opening and the balloon inflation opening.

In an aspect, alone or in combination with any other aspect, a balloon catheter comprises a catheter having longitudinally opposite proximal and distal catheter end portions. The distal catheter end portion includes a primary catheter branch and a secondary catheter branch each extending longitudinally from the proximal catheter end portion. The primary catheter branch has a distal catheter sidewall opening. The secondary catheter branch has a catheter inflation opening. An expandable balloon is positioned on at least the distal catheter end portion. The balloon defines at least one balloon opening aligned and in fluid communication with the distal catheter sidewall opening. A first balloon catheter lumen extends substantially longitudinally from the proximal catheter end portion to a distal catheter tip on the primary catheter branch end portion. A second balloon catheter lumen extends substantially longitudinally from the proximal catheter end portion to at least the balloon opening. An inflation lumen extends substantially longitudinally from the proximal catheter end portion to the catheter inflation opening in the secondary catheter branch. The catheter inflation opening provides fluid communication between the inflation lumen and an inflatable volume of the expandable balloon.

In an aspect, alone or in combination with any other aspect, a balloon catheter comprises a catheter having longitudinally opposite proximal and distal catheter end portions. The distal catheter end portion has a distal catheter tip. The catheter has an open catheter slit extending from the distal catheter tip toward the proximal catheter end portion. The catheter has a catheter inflation opening. An expandable balloon is positioned on at least the distal catheter end portion. The balloon defines at least one balloon opening aligned with the open catheter slit. A fixer at least partially attaches the expandable balloon to an inner catheter surface of the catheter. The fixer has longitudinally opposite proximal and distal fixer tips and at least one fixer sidewall opening spaced longitudinally from each of the proximal and distal fixer tips. The fixer is disposed within the catheter such that the fixer is at least partially surrounded by the inner catheter surface and the at least one fixer sidewall opening is aligned with both the open catheter slit and the at least one balloon opening. A first balloon catheter lumen extends substantially longitudinally from the proximal catheter end portion to the distal catheter tip. A second balloon catheter lumen extends substantially longitudinally from the proximal catheter end portion to at least the fixer sidewall opening. An inflation lumen extends substantially longitudinally from the proximal catheter end portion to the catheter inflation opening. The catheter inflation opening provides fluid communication between the inflation lumen and an inflatable volume of the expandable balloon.

In an aspect, alone or in combination with any other aspect, a method for constructing a balloon catheter comprises providing a catheter. A balloon material is placed at least partially circumferentially about an outer catheter surface so that at least a portion of the balloon material is adjacent to at least a portion of an open catheter slit. At least a portion of the balloon material is urged through the open catheter slit and into an interior of the catheter. At least a portion of the balloon material inserted into the interior of the catheter is attached to at least a portion of the inner catheter surface. The balloon material forms an open balloon slit that is aligned with the open catheter slit when the balloon material is attached to the inner catheter surface. The balloon material defines first and second balloon end portions on opposing sides of an open balloon slit. A fixer is inserted through the open balloon slit and into the interior of the catheter. The fixer is disposed on a portion of the balloon material in the interior of the catheter. The fixer is aligned with a fixer sidewall opening being aligned with the open balloon slit. The fixer is attached to at least one of the inner catheter surface and the portion of the balloon material that is attached to the inner catheter surface. A portion of the first balloon end portion is attached to at least one of the fixer and the second balloon end portion such that the at least one balloon opening is defined between the first and second balloon end portions. The at least one balloon opening is aligned with the at least one fixer sidewall opening. At least a portion of a proximal balloon material end is circumferentially attached to at least a portion the outer catheter surface adjacent to the open catheter slit. At least a portion of a distal balloon material end is circumferentially attached to at least a portion the outer catheter surface adjacent to the open catheter slit.

### BRIEF DESCRIPTION OF THE DRAWINGS

For a better understanding, reference may be made to the accompanying drawings, in which:

FIG. 1 is a top view of a balloon catheter according to one aspect of the present invention, including the balloon catheter in an example configuration;

FIGS. 1a-j depict cross-sectional views of the aspect of FIG. 1 taken along lines 1a-1a, 1b-1b, 1c-1c, 1d-1d, 1e-1e, 1f-1f, 1g-1g, 1h-1h, 1i-1i, and 1j-1j, respectively;

FIG. 2 is a perspective side view of a component of the balloon catheter of FIG. 1;

FIGS. 2a-d depict cross-sectional views of the aspect of FIG. 2 taken along lines 2a-2a, 2b-2b, 2c-2c, and 2d-2d, respectively;

FIG. 3 is a schematic and cross-sectional side view of the component of FIG. 2;

FIG. 4 is a perspective rear view of a component of the balloon catheter of FIG. 1;

FIGS. 4a-c depict cross-sectional views of the aspect of FIG. 4 taken along lines 4a-4a, 4b-4b, and 4c-4c, respectively;

FIG. 5 is a schematic and cross-sectional side view of the component of FIG. 4;

FIG. 6 is a schematic and cross-sectional side view of a portion of the balloon catheter of FIG. 1;

FIGS. 6a-f depict cross-sectional views of the aspect of FIG. 6 taken along lines 6a-6a, 6b-6b, 6c-6c, 6d-6d, 6e-6e, and 6f-6f, respectively;

FIG. 7 is a schematic and cross-sectional side view of a component of the balloon catheter of FIG. 1, including the component in a first condition;

FIGS. 7a-e depict cross-sectional views of the aspect of FIG. 7 taken along lines 7a-7a, 7b-7b, 7c-7c, 7d-7d, and 7e-7e, respectively;

FIG. 8 is a schematic and cross-sectional side view of the component of FIG. 7, including the component in a second condition;

FIGS. 8a-e depict cross-sectional views of the aspect of FIG. 8 taken along lines 8a-8a, 8b-8b, 8c-8c, 8d-8d, and 8e-8e, respectively;

FIG. 9 is a schematic and cross-sectional side view of the balloon catheter of FIG. 1;

FIGS. 9a-i depict cross-sectional views of the aspect of FIG. 9 taken along lines 9a-9a, 9b-9b, 9c-9c, 9d-9d, 9e-9e, 9f-9f, 9g-9g, 9h-9h, and 9i-9i, respectively;

FIGS. 10-19 schematically illustrate an example sequence of constructing a portion of the aspect of FIG. 1;

FIG. 20 is a perspective top view of the balloon catheter of FIG. 1 having an additional component;

FIGS. 20a-c depict cross-sectional views of the aspect of FIG. 20 taken along lines 20a-20a, 20b-20b, and 20c-20c, respectively;

FIGS. 21-24 schematically illustrate an example sequence of operation of a portion of the aspect of FIG. 1, including cross-sectional views taken along lines 22a-22a, 22b-22b, 22c-22c, 22d-22d, 23a-23a, 23b-23b, 23c-23c, and 23d-23d, respectively;

FIGS. 25-27 schematically illustrate an example sequence of operation of a portion of the aspect of FIG. 1, including cross-sectional views taken along lines 25a-25a, 25b-25b, 25c-25c, 25d-25d, 26a-26a, 26b-26b, 26c-26c, 26d-26d, 27a-27a, 27b-27b, and 27c-27c, respectively;

FIGS. 28-30 schematically illustrate an example sequence of operation of a portion of the aspect of FIG. 1;

FIG. 31 is a schematic and cross-sectional side view of the component of FIG. 2 in an example configuration;

FIGS. 31a-f depict cross-sectional views of the aspect of FIG. 31 taken along lines 31a-31a, 31b-31b, 31c-31c, 31d-31d, 31e-31e, and 31f-31f, respectively;

FIG. 32 is a perspective side view of the component of FIG. 2 in an example configuration;

FIG. 33 is a perspective side view of the component of FIG. 4 in an example configuration;

FIG. 34 is a perspective side view of the balloon catheter of FIG. 1 having the components of FIGS. 32-33;

FIG. 35 is a perspective side view of the component of FIG. 4 in example configuration;

FIG. 36 is a cross-sectional view of the balloon catheter of FIG. 1 in an example configuration having the component of FIG. 35;

FIG. 37 is a perspective side view of the component of FIG. 4 in example configuration;

FIG. 38 is a cross-sectional view of the balloon catheter of FIG. 1 in an example configuration having the component of FIG. 37;

FIG. 39 is a perspective side view of the component of FIG. 4 in example configuration;

FIG. 40 is a perspective top view of the balloon catheter of FIG. 1 in an example configuration having the component of FIG. 39;

FIG. 41 is a perspective top view of a component of the balloon catheter of FIG. 1 in an example configuration;

FIG. 42 is a perspective top view of the balloon catheter of FIG. 1 in an example configuration having the component of FIG. 41;

FIG. 43 depicts the balloon catheter of the aspect of FIG. 42 in an example use environment;

FIG. 44 depicts the balloon catheter of the aspect of FIG. 1 in an example use environment;

FIG. 45 depicts the balloon catheter of the aspect of FIG. 42 in an example use environment;

FIGS. 45a-c schematically illustrate an example sequence of operation of portion X of the aspect of FIG. 45;

FIG. 46 depicts the balloon catheter of the aspect of FIG. 42 in an example use environment;

FIGS. 46a-d schematically illustrate an example sequence of operation of portion Y of the aspect of FIG. 46;

FIG. 47 is a perspective top view of the additional component of FIG. 20 in an example configuration;

FIG. 48 is a perspective top view of the additional component of FIG. 20 in an example configuration; and

FIG. 49 is a perspective top view of the additional component of FIG. 20 in an example configuration.

FIGS. 50-56 schematically illustrate an example sequence of constructing a portion of the balloon catheter aspect of FIG. 1, including the balloon catheter in an example configuration;

FIG. 57 is a schematic and partial cross-sectional side view of the balloon catheter of FIG. 56;

FIGS. 57a-e depict cross-sectional views of the aspect of FIG. 57 taken along lines 57a-57a, 57b-57b, 57c-57c, 57d-57d, and 57e-57e, respectively;

FIG. 58 depicts the cross-sectional view of FIG. 57b in a second condition;

FIG. 59 depicts the cross-sectional view of FIG. 57d in a second condition;

FIGS. 60-69 schematically illustrate an example sequence of constructing a portion of the balloon catheter aspect of FIG. 1, including the balloon catheter in an example configuration;

FIGS. 69a-e depict cross-sectional views of the aspect of FIG. 69 taken along lines 69a-69a, 69b-69b, 69c-69c, 69d-69d, and 69e-69e, respectively;

FIG. 70 depicts the cross-sectional view of FIG. 69b in a second condition;

FIG. 71 depicts the cross-sectional view of FIG. 69c in a second condition;

FIG. 72 depicts the cross-sectional view of FIG. 69d in a second condition;

FIGS. 73-81 schematically illustrate an example sequence of constructing a portion of the balloon catheter aspect of FIG. 1, including the balloon catheter in an example configuration, and including cross-sectional views taken along lines 73a-73a, 73b-73b, 73c-73c, 73d-73d, 74a-74a, 74b-74b, 74c-74c, 74d-74d, 75a-75a, 75b-75b, 75c-75c, 75d-75d, 76a-76a, 76b-76b, 76c-76c, 76d-76d, 77a-77a, 77b-77b, 77c-77c, 77d-77d, 78a-78a, 78b-78b, 78c-78c, and 78d-78d, respectively;

FIG. 82 is a schematic and cross-sectional side view of the balloon catheter of FIG. 81;

FIGS. 82a-h depict cross-sectional views of the aspect of FIG. 82 taken along lines 82a-82a, 82b-82b, 82c-82c, 82d-82d, 82e-82e, 82f-82f, 82g-82g, and 82h-82h, respectively;

FIG. 83 depicts the cross-sectional view of FIG. 82b in a second condition;

FIG. 84 depicts the cross-sectional view of FIG. 82c in a second condition;

FIG. 85 depicts the cross-sectional view of FIG. 82e in a second condition;

FIG. 86 depicts the cross-sectional view of FIG. 82f in a second condition;

FIG. 87 depicts the cross-sectional view of FIG. 82g in a second condition;

FIG. 88 is a perspective side view of the balloon catheter aspect of FIG. 1, including the balloon catheter in an example configuration;

FIGS. 88a-f depict cross-sectional views of the aspect of FIG. 1 taken along lines 88a-88a, 88b-88b, 88c-88c, 88d-88d, 88e-88e, and 88f-88f, respectively; and

FIG. 89 is a perspective side view of a component of the balloon catheter of FIG. 88.

### DESCRIPTION OF ASPECTS OF THE DISCLOSURE

As used herein, the term “user” can be used interchangeably to refer to an individual who prepares for, assists with, and/or performs the operation of a tool, and/or to an individual who prepares for, assists with, and/or performs a procedure.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” can be interpreted to include X and Y.

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

It will be understood that when an element is referred to as being “on,” “attached” to, etc., another element, it can be directly on or attached to the other element or intervening elements may also be present. It will also be appreciated by those of skill in the art that references to a structure or feature that is disposed “directly adjacent” another feature may have portions that overlap or underlie the adjacent feature, whereas a structure or feature that is disposed “adjacent” another feature may not have portions that overlap or underlie the adjacent feature.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or Figures unless specifically indicated otherwise.

The invention comprises, consists of, or consists essentially of the following features, in any combination.

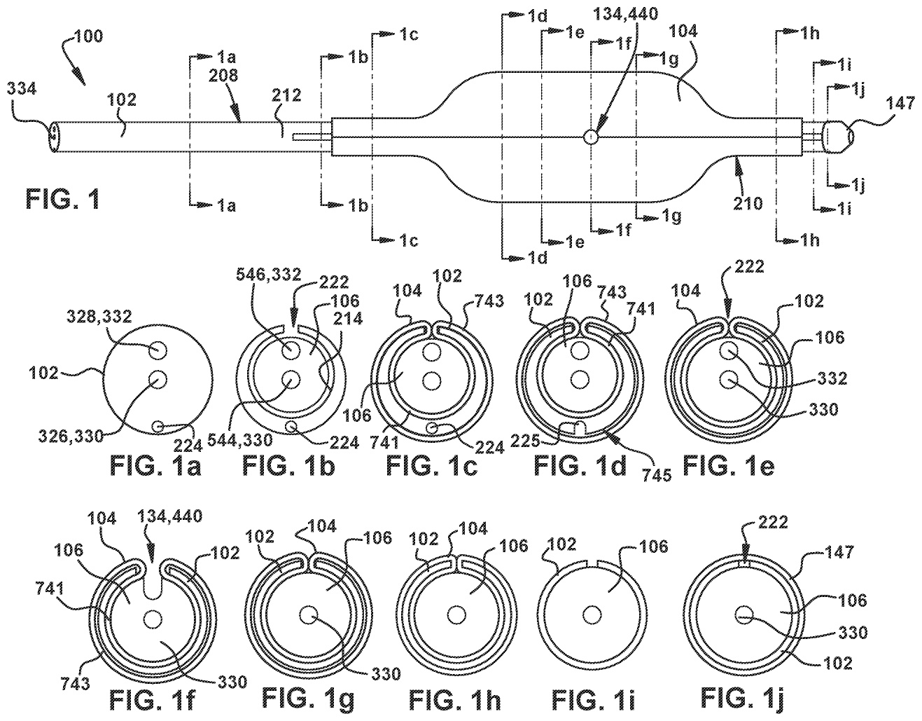

FIG. 1 depicts one configuration of a balloon catheter 100. The balloon catheter 100 includes a catheter 102, an expandable balloon 104, and a fixer 106. FIGS. 1a-j depict cross-sectional views taken at various locations along the balloon catheter 100, to show the arrangement of at least the catheter 102, the expandable balloon 104, and the fixer 106.

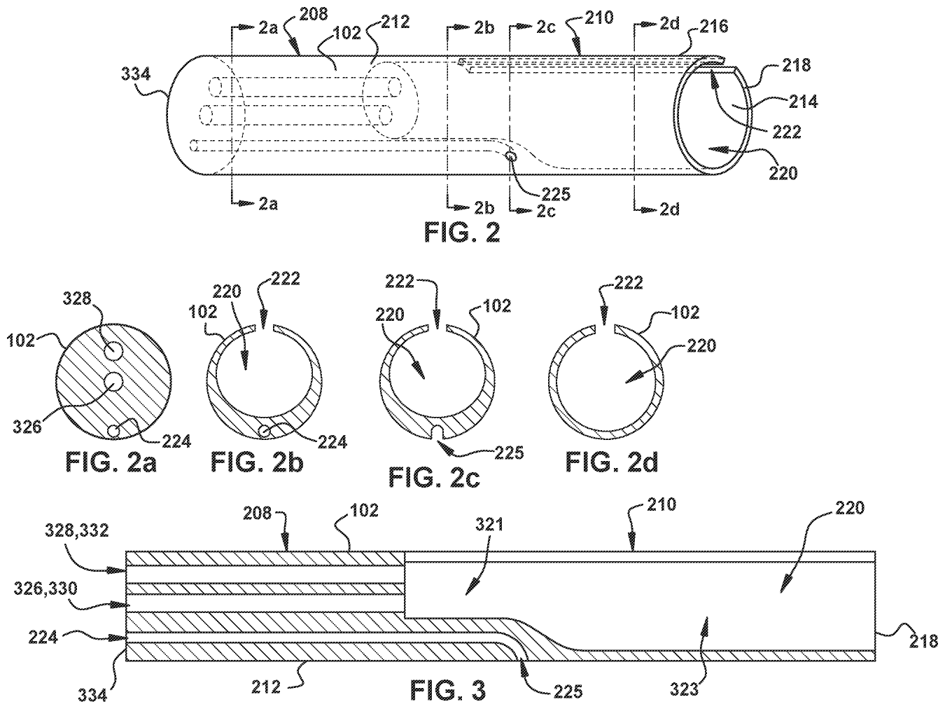

As shown in FIGS. 2-3, the catheter 102 has longitudinally opposite proximal and distal catheter end portions 208, 210 and a catheter sidewall 212 extending longitudinally between the proximal and distal catheter end portions 208, 210. The term “longitudinal” is used herein to indicate a substantially horizontal direction, in the orientation of FIGS. 2-3. The catheter sidewall 212 includes radially opposing inner and outer catheter surfaces 214, 216. The term “radial” is used herein to indicate a direction substantially perpendicular to the “longitudinal” direction. The inner catheter surface 214 extends longitudinally from a distal catheter tip 218 to the proximal catheter end portion 208. The inner catheter surface 214 at least partially surrounds and defines a distal catheter lumen 220 that extends longitudinally from the proximal catheter end portion 208 to the distal catheter tip 218. The distal catheter lumen 220 includes first and second distal catheter lumen portions 321, 323. The first distal catheter lumen portion 321 is between the proximal catheter end portion 208 and the second distal catheter lumen portion 323. The second distal catheter lumen portion 323 extends further in the radial direction than the first distal catheter lumen portion 321 so that the second distal catheter lumen portion 323 is larger than the first distal catheter lumen portion 321. An open catheter slit 222 extends longitudinally from the distal catheter tip 218 toward the proximal catheter end portion 208, and radially through the catheter sidewall 212. FIGS. 2a-d depict cross-sectional views taken at various locations along the catheter 102.

The proximal catheter end portion 208 may include a plurality of lumens extending substantially longitudinally therethrough. For example, as shown in FIGS. 2-2c, the proximal catheter end portion 208 includes an inflation lumen 224. The inflation lumen 224 may extend substantially longitudinally from the proximal catheter end portion 208 to a catheter inflation opening 225 in the catheter sidewall 212. The catheter inflation opening 225 is in fluid communication with an inflatable volume of the balloon 104, so that the balloon 104 may be inflated via an outside fluid source (i.e., a liquid or gas source) that selectively directs fluid through the inflation lumen 224. The inflation lumen 224 may be placed in fluid communication with the outside fluid source in any desired manner.

As shown in FIG. 3, first portions 326, 328 of first and second balloon catheter lumens 330, 332 extend substantially longitudinally from a proximal catheter tip 334 to the distal catheter lumen 220. As shown in FIGS. 2-3, prior to the full assembly of the balloon catheter 100, the first portions 326, 328 of the first and second balloon catheter lumens 330, 332 are in fluid communication with the distal catheter lumen 220.

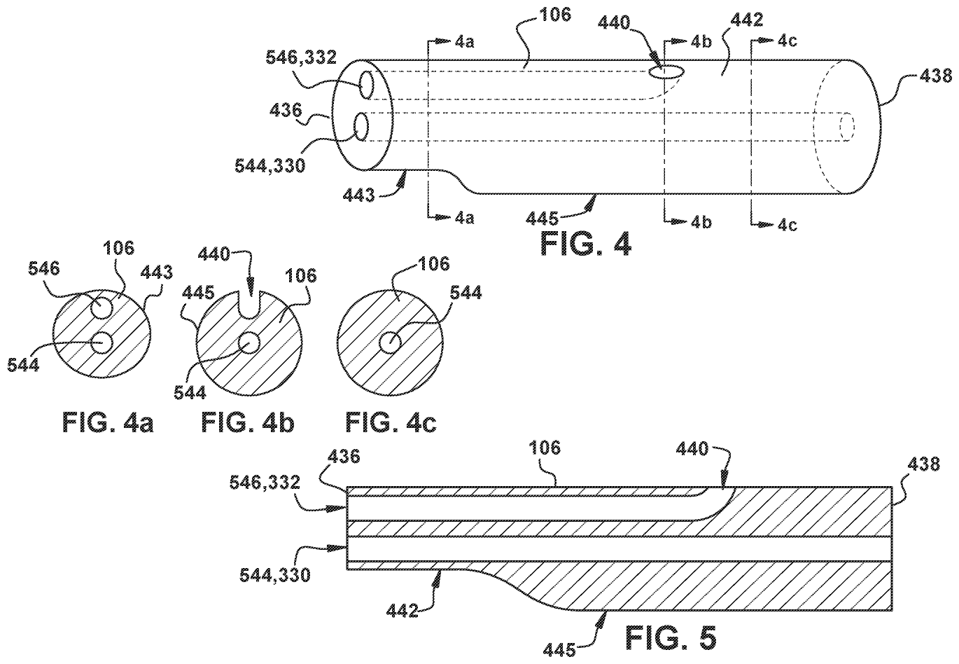

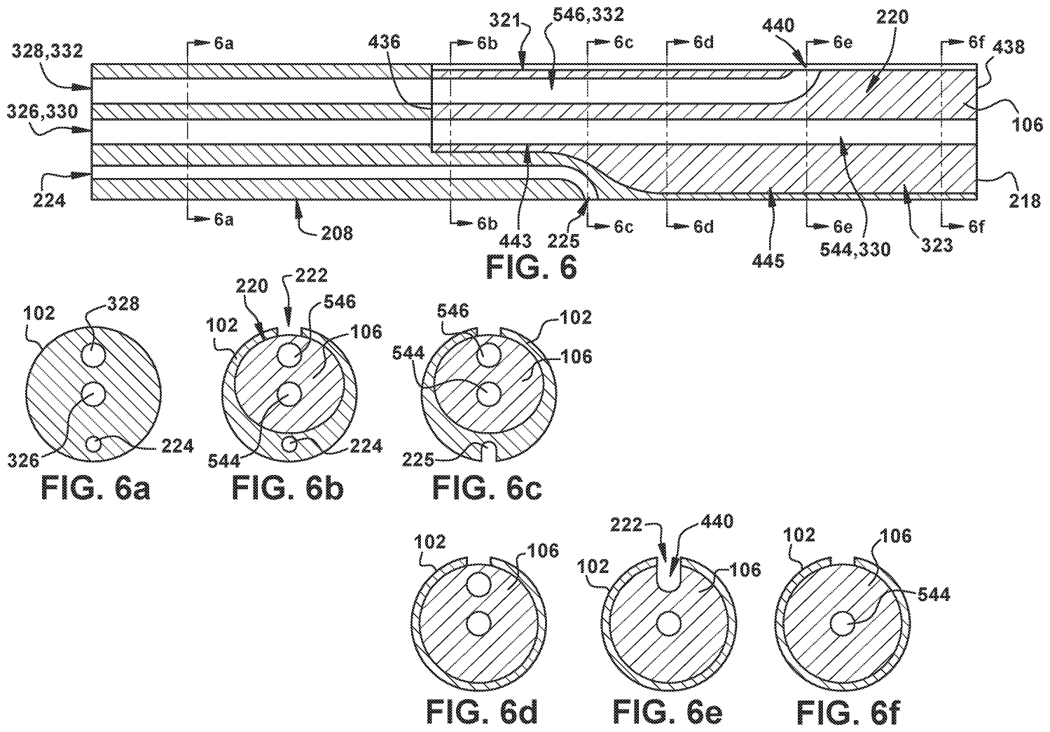

As will be described in more detail below, the fixer 106 at least partially attaches the balloon 104 to the inner catheter surface 214. As shown in FIGS. 1-1j, the fixer 106 is disposed within the distal catheter lumen 220 such that the fixer 106 is at least partially surrounded by the inner catheter surface 214. As shown in FIGS. 4-5, the fixer 106 has longitudinally opposite proximal and distal fixer tips 436, 438 and a fixer sidewall opening 440 extending radially through a fixer sidewall 442. The fixer sidewall opening 440 is spaced longitudinally from each of the proximal and distal fixer tips 436, 438. The fixer 104 also includes proximal and distal fixer end portions 443, 445. The proximal fixer end portion 443 includes the proximal fixer tip 436 and extends longitudinally from the proximal fixer tip 436 to the distal fixer end portion 445. The distal fixer end portion 445 includes the distal fixer tip 438. The distal fixer end portion 445 also extends further in the radial direction than the proximal fixer end portion 443 so that the distal fixer end portion 445 is larger than the proximal fixer end portion 443. As shown in FIGS. 1 and 6-6f, when the balloon catheter 100 is at least partially assembled, the fixer sidewall opening 440 is aligned and in fluid communication with the open catheter slit 222 and a balloon opening 134. Further, when at least partially assembled, the proximal fixer end portion 443 is positioned in the first distal catheter lumen portion 321, while the distal fixer end portion 445 is positioned in the second distal catheter lumen portion 323. FIGS. 6a-f depict cross-sectional views taken at various locations along the partially assembled balloon catheter 100, to show the arrangement of the catheter 102 and the fixer 106.

As shown in FIGS. 5-6, a second portion 544 of the first balloon catheter lumen 330 extends substantially longitudinally from the proximal fixer tip 436 to the distal fixer tip 438, while a second portion 546 of the second balloon catheter lumen 332 extends substantially longitudinally from the proximal fixer tip 436 to the fixer sidewall opening 440. Accordingly, when the balloon catheter 100 is at least partially assembled, the fixer 106 is positioned in the distal catheter lumen 220 such that the first and second portions 326, 544 of the first balloon catheter lumen 330 are aligned and in fluid communication with one another, and the first and second portions 328, 546 of the second balloon catheter lumen 332 are aligned and in fluid communication with one another. As such, the first balloon catheter lumen 330 extends substantially longitudinally from the proximal catheter tip 334 to the distal fixer tip 438, while the second balloon catheter lumen 332 extends substantially longitudinally from the proximal catheter tip 334 to the fixer sidewall opening 440. As shown in FIG. 6, the distal fixer tip 438 may be longitudinally aligned with the distal catheter tip 218 and, thus, the first balloon catheter lumen 330 may also be said to extend from the proximal catheter tip 334 to the distal catheter tip 218.

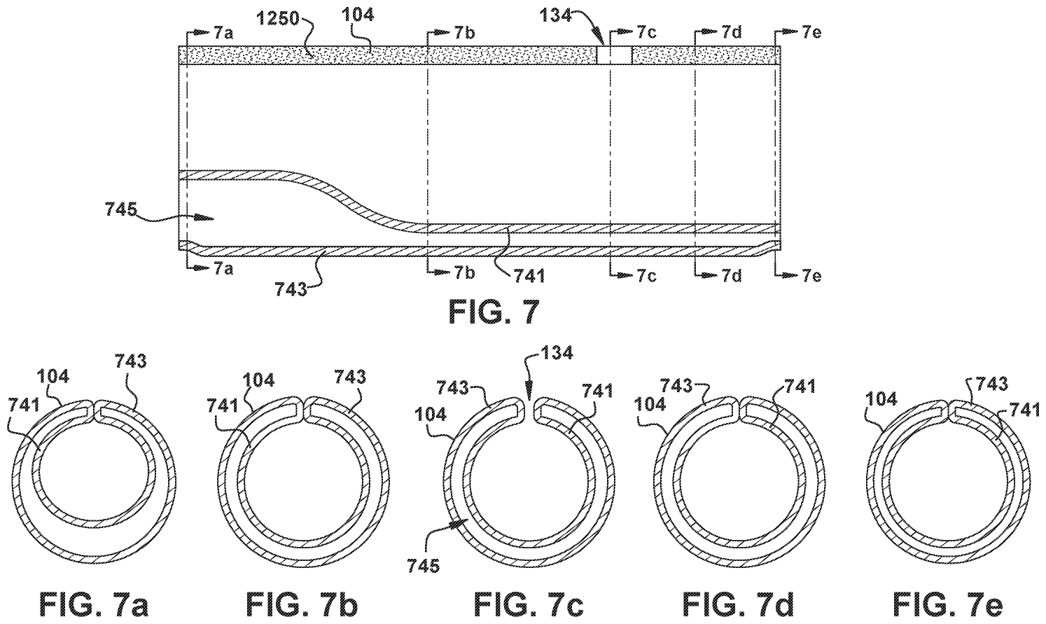

As shown in FIGS. 1-1j and FIGS. 7-7e, the balloon 104 may be positioned on at least the distal catheter end portion 210. It is contemplated, however, that at least a portion of the balloon 104 may be positioned on the proximal catheter end portion 208. The balloon 104 defines the radially extending balloon opening 134 that is aligned and in fluid communication with the open catheter slit 222. The balloon opening 104 is also aligned with the fixer sidewall opening 440 and, thus, the second balloon catheter lumen 332 may extend substantially longitudinally from the proximal catheter tip 334 to the balloon opening 134.

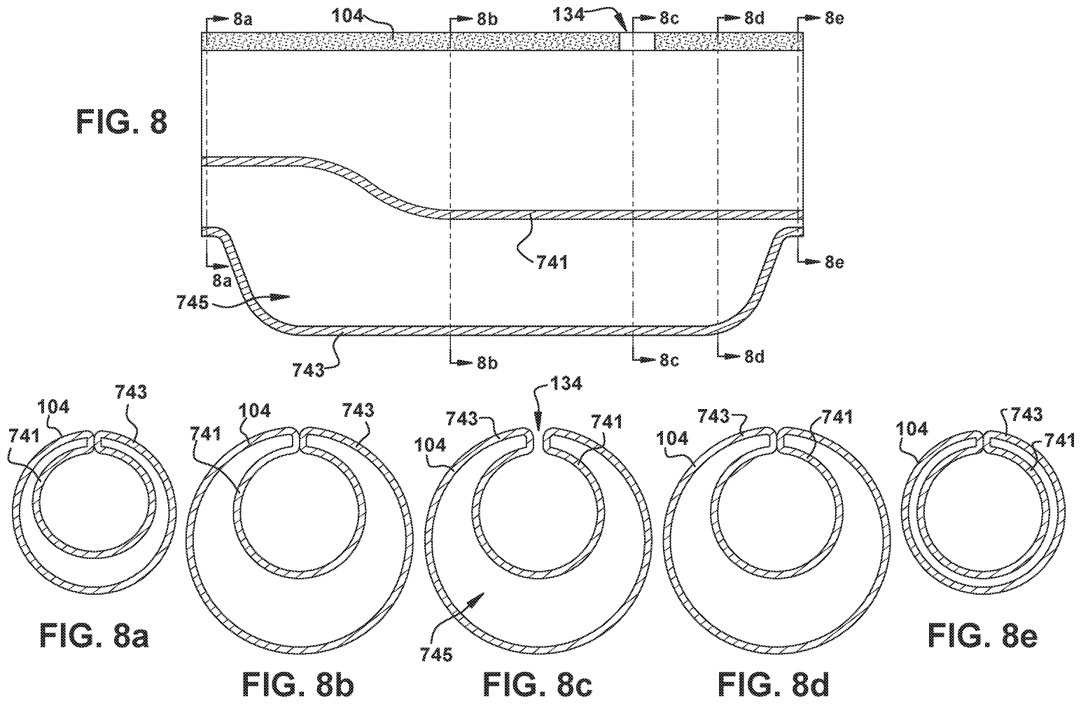

The balloon 104 includes an inner balloon portion 741 fixed between the inner catheter surface 214 and the fixer 106 and an outer balloon portion 743 surrounding a portion of the outer catheter surface 216, with the inflatable volume 745 of the balloon 104 being between the two portions 741, 743. FIGS. 7-7e show the balloon 104 in an uninflated or collapsed condition, while FIGS. 8-8e show the balloon 104 in an inflated or expanded condition. As shown in FIGS. 7-8e, the balloon 104 may be configured such that only the outer balloon portion 743 moves during inflation of the balloon 104 because the inner balloon portion 741 is fixed between the inner catheter surface 214 and the fixer 106.

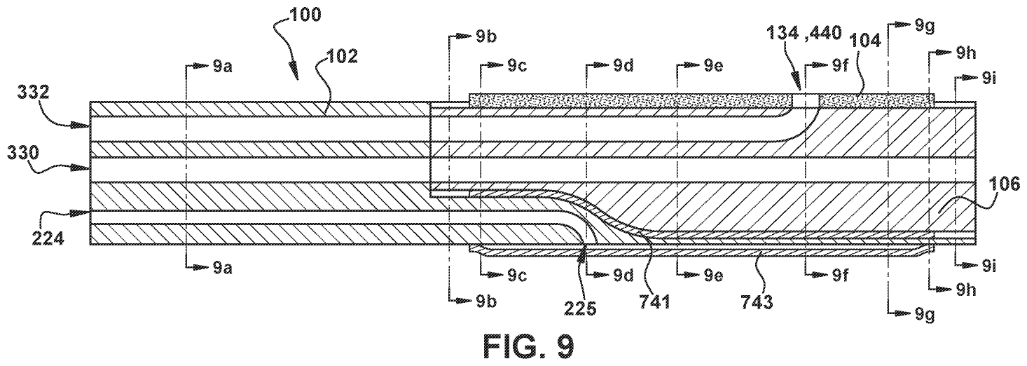

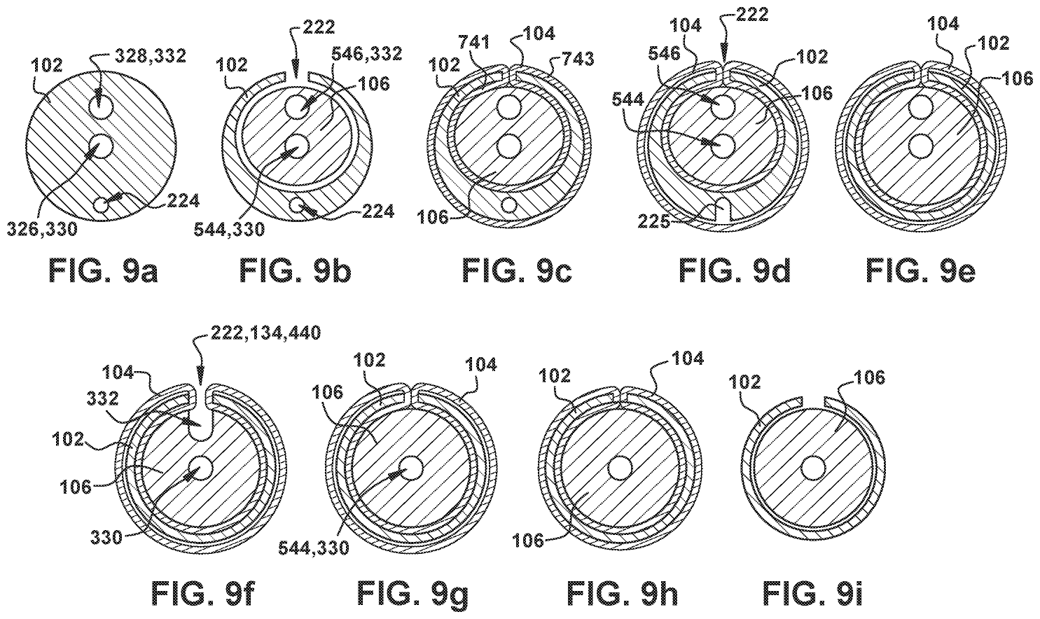

FIG. 9 schematically illustrates the balloon catheter 100 having with the catheter 102, the balloon 104, and the fixer 106 joined together. FIGS. 9a-i depict cross-sectional views taken at various locations along the balloon catheter 100, to show the arrangement of at least the catheter 102, the expandable balloon 104, and the fixer 106.

As shown in FIG. 1, the balloon catheter 100 may include a nosecone 147 attached (e.g., via an adhesive) to the distal catheter end portion 210. A “nosecone,” as used herein, is a structure resembling a cone, or, in other words, resembling a solid bounded by a circular or other closed plane base and the surface formed by line segments joining every point of the boundary of the base to a common vertex. The nosecone 147 may point, or narrow, in the longitudinally distal direction (shown as an arrow “D” in FIG. 1). The nosecone 147 may be used for a smooth atraumatic transition of the balloon catheter 100 into a target patient tissue site. The nosecone 147 may also extend the first balloon catheter lumen 330 so that the first balloon catheter lumen 330 extends substantially longitudinally from the proximal fixer tip 436 through the nosecone 147.

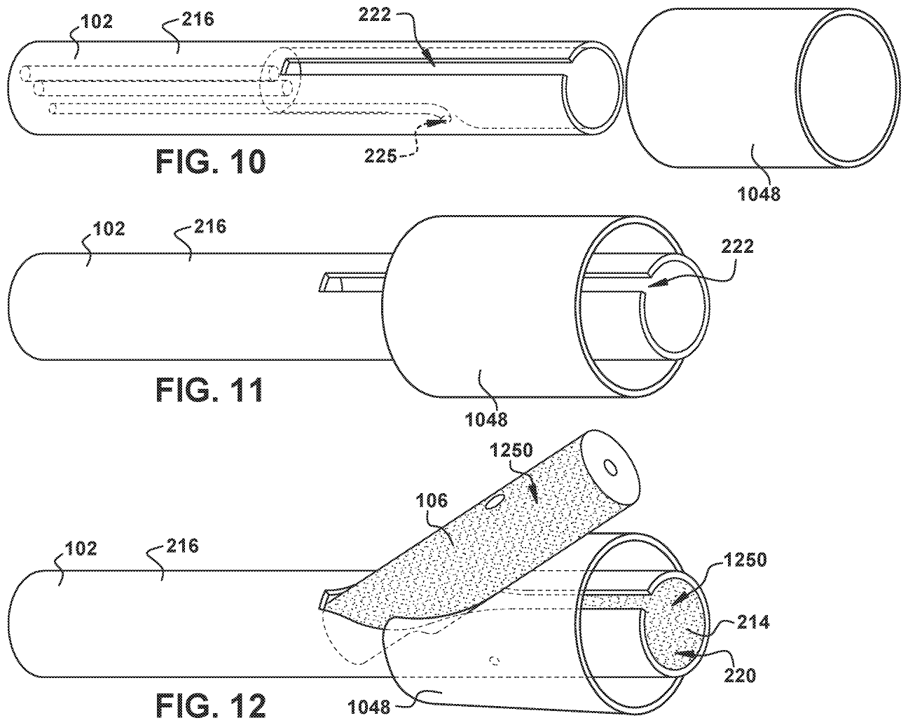

To construct the balloon catheter 100 described above, the catheter 102 is provided. As shown in FIGS. 10-11, a balloon material 1048 may be placed at least partially circumferentially about the outer catheter surface 216 so that at least a portion of the balloon material 1048 is radially adjacent to at least a portion of the open catheter slit 222. As shown in FIG. 12, an adhesive 1250 may be applied to at least one of the balloon material 1048, the inner catheter surface 214, and the outer surface of the fixer 106. The adhesive 1250 may be applied before, as, or after the balloon material 1048 is placed about the outer catheter surface 216.

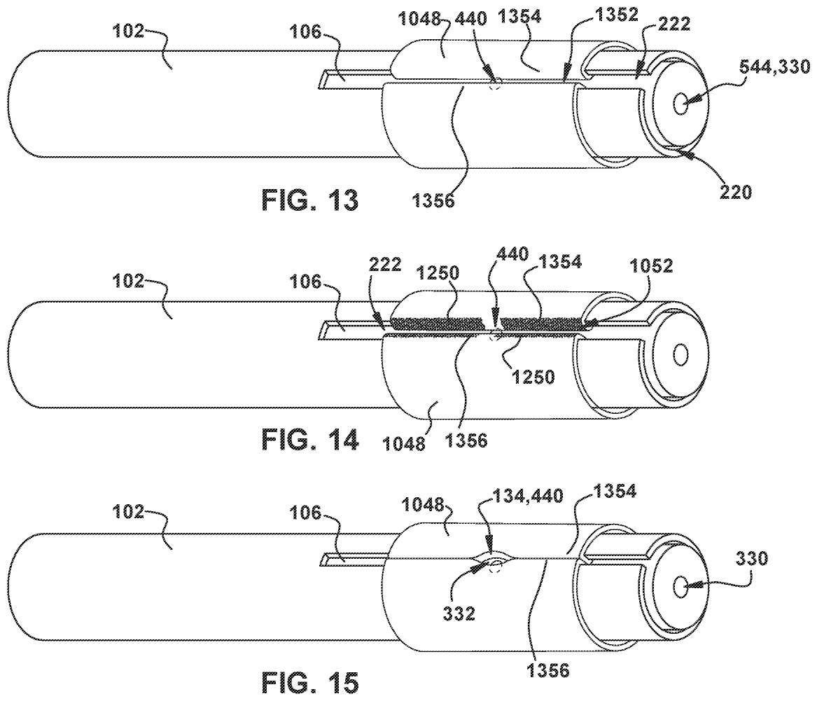

As shown in FIGS. 12-13, with the balloon material 1048 in place, the fixer 106 may be inserted through the open catheter slit 222 and into the distal catheter lumen 220 (i.e., into the interior of the catheter 102). As the fixer 106 is inserted through the open catheter slit 222, the fixer 106 is disposed on the balloon material 1048 and urges at least a portion of the balloon material 1048 through the open catheter slit 222 and into the distal catheter lumen 220. The fixer 106 thus is disposed on a portion of the balloon material 1048 after being inserted into the catheter 102. The fixer 106 is aligned with fixer sidewall opening 440 aligned with the open catheter slit 222. The fixer 106 is also aligned in the manner described above so that the first balloon catheter lumen 330 extends substantially longitudinally from the proximal catheter tip 334 to the distal fixer tip 438, and the second balloon catheter lumen 332 extends substantially longitudinally from the proximal catheter tip 334 to the fixer sidewall opening 440.

The fixer 106, the portion of the balloon material 1048 inserted into the interior of the catheter 102, and the inner catheter surface 214 are attached to one another via the adhesive 1250. Although each of the fixer 106, the balloon material 1048, and the inner catheter surface 214 is shown as being attached to each of the other of the fixer 106, the balloon material 1048, and the inner catheter surface 214, it is contemplated that at least one of the fixer 106, the balloon material 1048, and the inner catheter surface 214 may be attached to only one other of the fixer 106, the balloon material 1048, and the inner catheter surface 214. Further, although the balloon material 1048 is shown as being urged into the interior of the catheter 102 together with the fixer 106, the balloon material 1048 may be inserted into the interior of the catheter 102 prior to the insertion of the fixer 106.

As shown in FIG. 13, the balloon material 1048 may form an open balloon slit 1352 that is aligned with the open catheter slit 222 when the balloon material 1048 is attached to the inner catheter surface 214. The fixer 106 is also aligned with fixer sidewall opening 440 aligned with the open balloon slit 1352. The balloon material 1048 defines first and second balloon end portions 1354, 1356 on opposing sides of the open balloon slit 1352.

As shown in FIG. 14, an adhesive 1250 may be applied to at least one of the fixer 106 and the balloon material 1048 on or adjacent to the first and second balloon end portions 1354, 1356. As shown in FIGS. 14-15, via the adhesive 1250, a portion of the first balloon end portion 1354 is attached to at least one of the fixer 106 and the second balloon end portion 1356 in such a manner that the balloon opening 134 is defined between the first and second balloon end portions 1354, 1356. The second balloon end portion 1356 may also be attached to the fixer 106 via the adhesive 1250. When attached as described above, the at least one balloon opening 134 is aligned with the fixer sidewall opening 440 such that the second balloon catheter lumen 332 now extends from the proximal catheter tip 334 to the balloon opening 134.

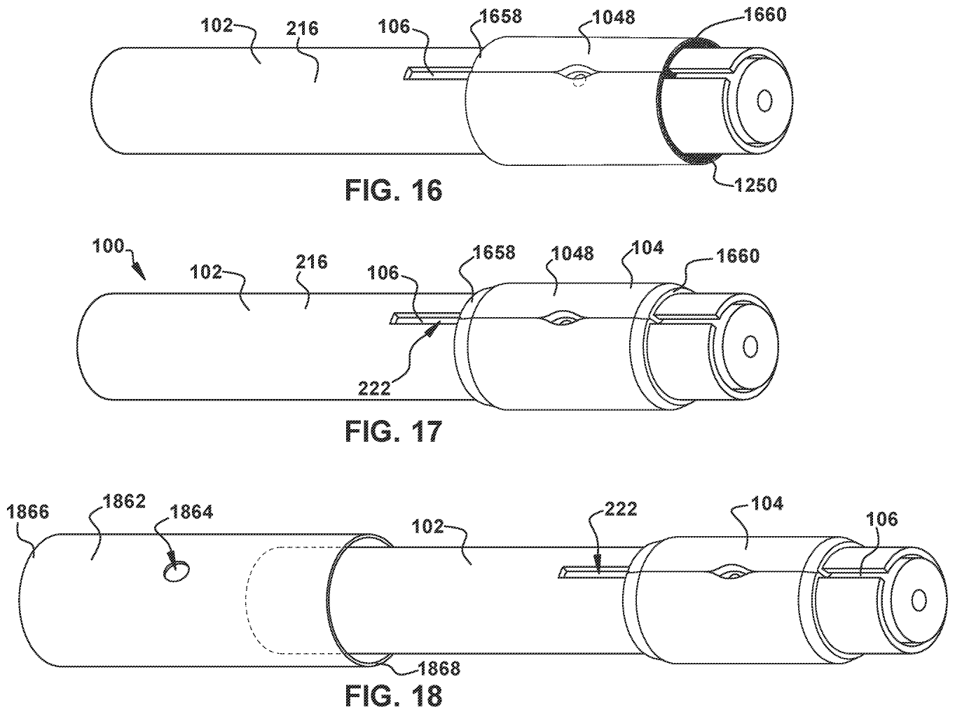

As shown in FIGS. 16-17, at least a portion of a proximal balloon material end 1658 is circumferentially attached to at least a portion of the outer catheter surface 216 adjacent to the open catheter slit 222 via an adhesive 1250. At least a portion of a distal balloon material end 1660 is also circumferentially attached to at least a portion of the outer catheter surface 216 adjacent to the open catheter slit 222 via an adhesive 1250. At least one of the proximal and distal balloon material ends 1658, 1660 may also be attached to the fixer 106. The expandable balloon 104 is formed having the balloon opening 134 once the balloon material 1048 is attached as described above.

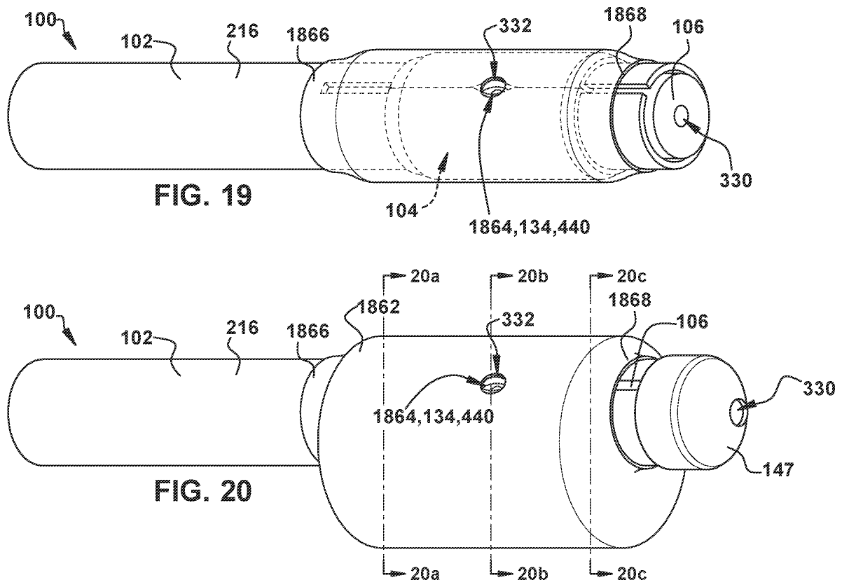

As shown in FIGS. 18-19, if desired, a wrap 1862 may be provided and placed at least partially circumferentially about the balloon 104, at least a portion of the fixer 106, and at least a portion of the catheter 102. The wrap 1862 is then aligned with a wrap opening 1864 aligned with the balloon opening 134 and the fixer sidewall opening 440 such that the second balloon catheter lumen 332 now extends from the proximal catheter tip 334 to the wrap opening 1864. Once aligned, proximal and distal wrap ends 1866, 1868 are secured (e.g., via an adhesive) to at least one of the outer catheter surface 216 and the fixer 106. If desired, a portion of the wrap 1862 may also or instead be attached (e.g., via an adhesive) to the balloon 104.

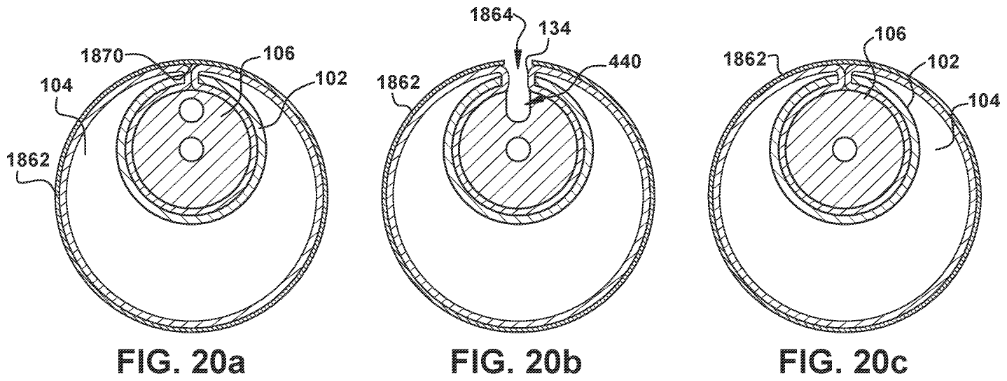

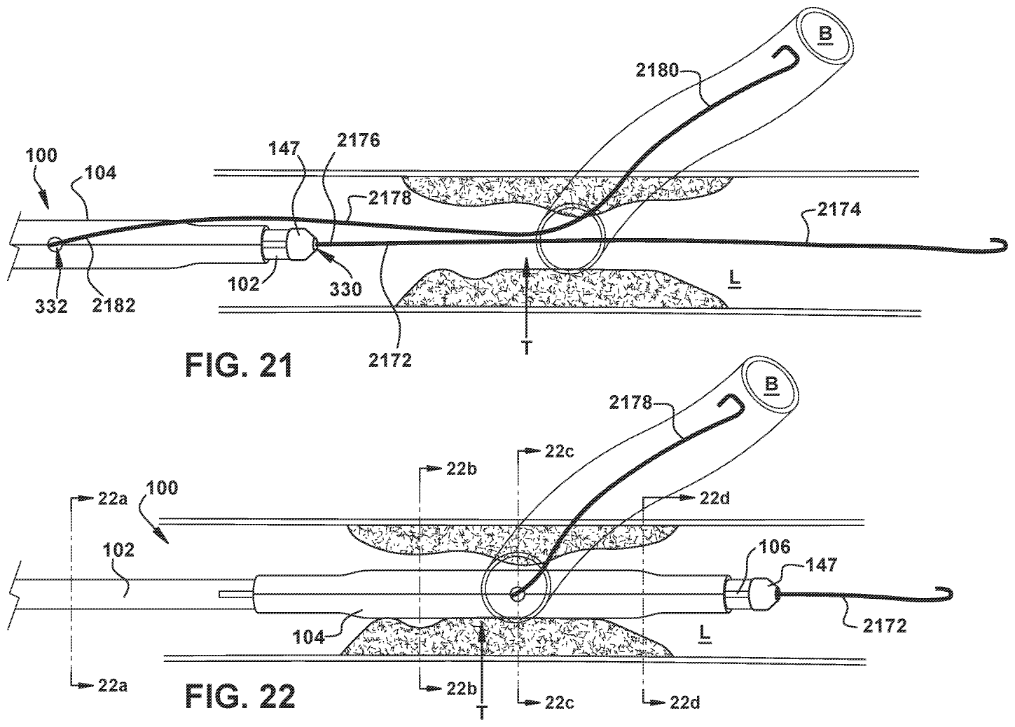

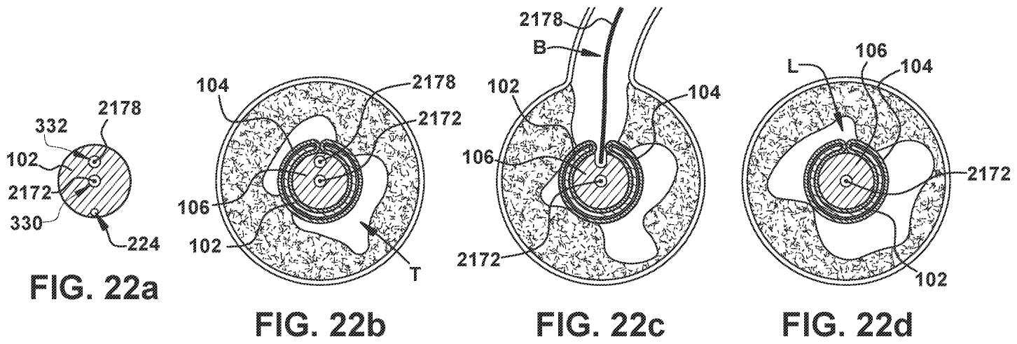

As shown in FIGS. 20-20c, the wrap 1862, when provided, encapsulates the balloon 104 between an inner cover surface 1870 of the wrap 1862 and at least one of the catheter 102 and the fixer 106. The wrap opening 1864 extends radially through the wrap 1862 and is configured to be aligned and in fluid communication with the balloon opening 134 and the fixer sidewall opening 440. As shown in FIGS. 20a and 20c, when the balloon 104 is in an expanded condition, the wrap 1862 may help form a more rounded or circular cross-section for the balloon catheter 100 in the area of the expanded balloon 104.

In use, the balloon catheter 100, as previously described, is provided. The balloon catheter 100 may be at least partially assembled prior to use in the manner (or a similar manner to) the one described above. At least FIGS. 21-24 illustrate an example sequence of operation/use of the balloon catheter 100 depicts a balloon catheter 100 that is constructed without the wrap 1862. However, a balloon catheter 100 having the wrap 1862 may be used in a similar manner as described below.

FIGS. 21-24 depict an example sequence of operation of the balloon catheter 100. A first guidewire 2172 is inserted into a target patient tissue site T in a patient lumen L with a first guidewire distal end 2174 of the first guidewire 2172 being positioned in the patient lumen L. A second guidewire 2178 is inserted into the target patient tissue site T with a second guidewire distal end 2180 of the second guidewire 2178 being positioned in a patient lumen branch B that extends from the patient lumen L. A first guidewire proximal end 2176 of the first guidewire 2172 is directed through the first balloon catheter lumen 330, while a second guidewire proximal end 2182 of the second guidewire 2178 is directed through the second balloon catheter lumen 332. As shown in FIGS. 21-22, the balloon catheter 100 is directed to the target patient tissue site T along the first and second guidewires 2172, 2178.

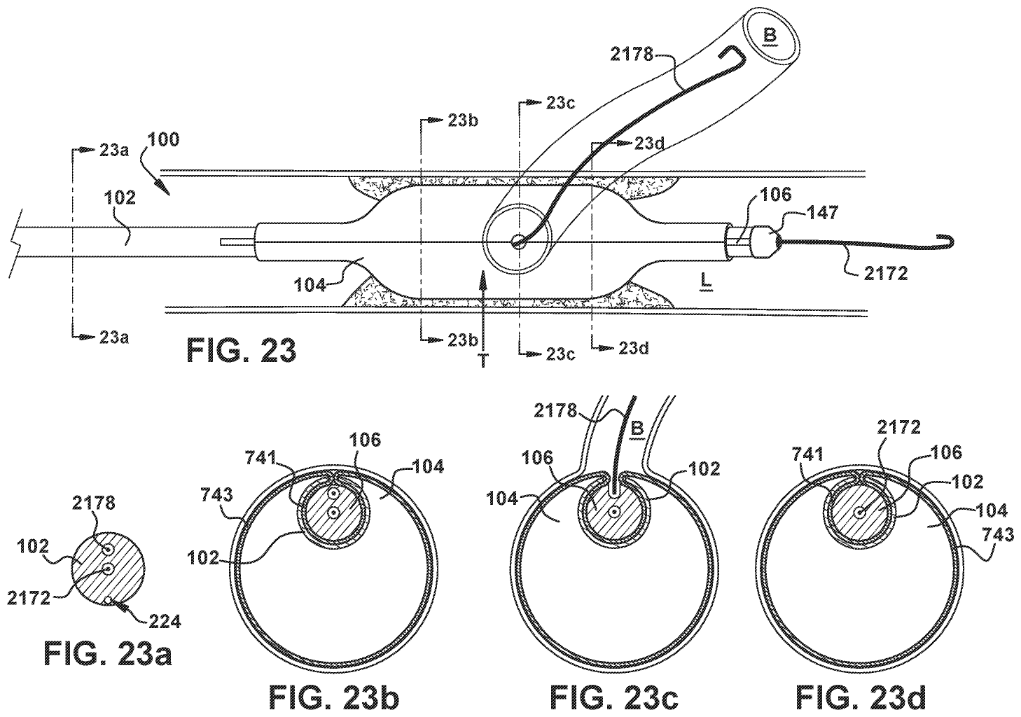

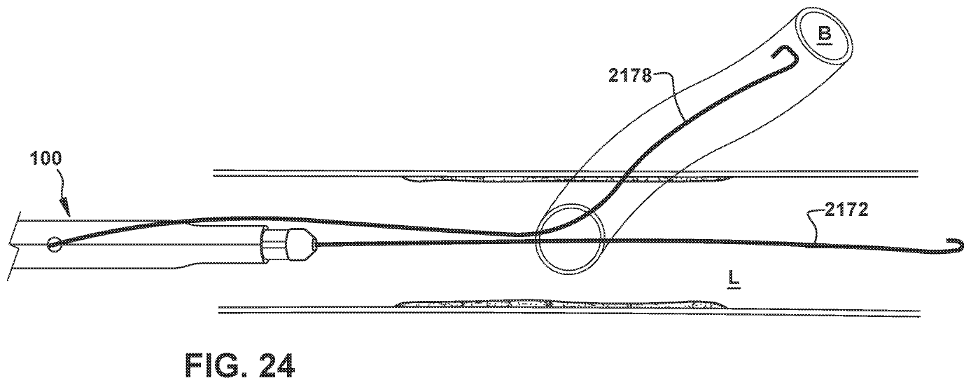

As shown in FIG. 22, the balloon catheter 100 is positioned at the target patient tissue site T with at least a portion of the catheter 102, at least a portion of the fixer 106, and at least a portion of the balloon 104 being positioned in the patient lumen L, and with the balloon opening 134 directed toward the patient lumen branch B. FIGS. 22a-d depict cross-sectional views taken at various locations along the balloon catheter 100, to show the arrangement of the balloon catheter 100, the first and second guidewires 2172, 2178, and the target patient tissue site T in FIG. 22. As shown in FIG. 23, with the balloon catheter 100 in position, the balloon 104 is inflated to dilate the patient lumen L at the target patient tissue site T. FIGS. 23a-d depict cross-sectional views taken at various locations along the balloon catheter 100, to show the arrangement of the balloon catheter 100, the first and second guidewires 2172, 2178, and the target patient tissue site T in FIG. 23. The balloon 104 is deflated after the patient lumen L at the target patient tissue site T has achieved a predetermined amount of dilation. As shown in FIG. 24, the balloon catheter 100 may then be removed from the target patient tissue site T, while the first and second guidewires 2172, 2178 are maintained substantially in position at the target patient site T. Because the first and second guidewires 2172, 2178 are maintained at the target patient tissue site T, medical instruments (such as, e.g., the balloon catheter 100 or other medical instruments) may be advanced along one or both of the first and second guidewires 2172, 2178 to perform a medical procedure in the patient lumen L, in the patient lumen branch B, and/or at or adjacent to the target patient tissue site T.

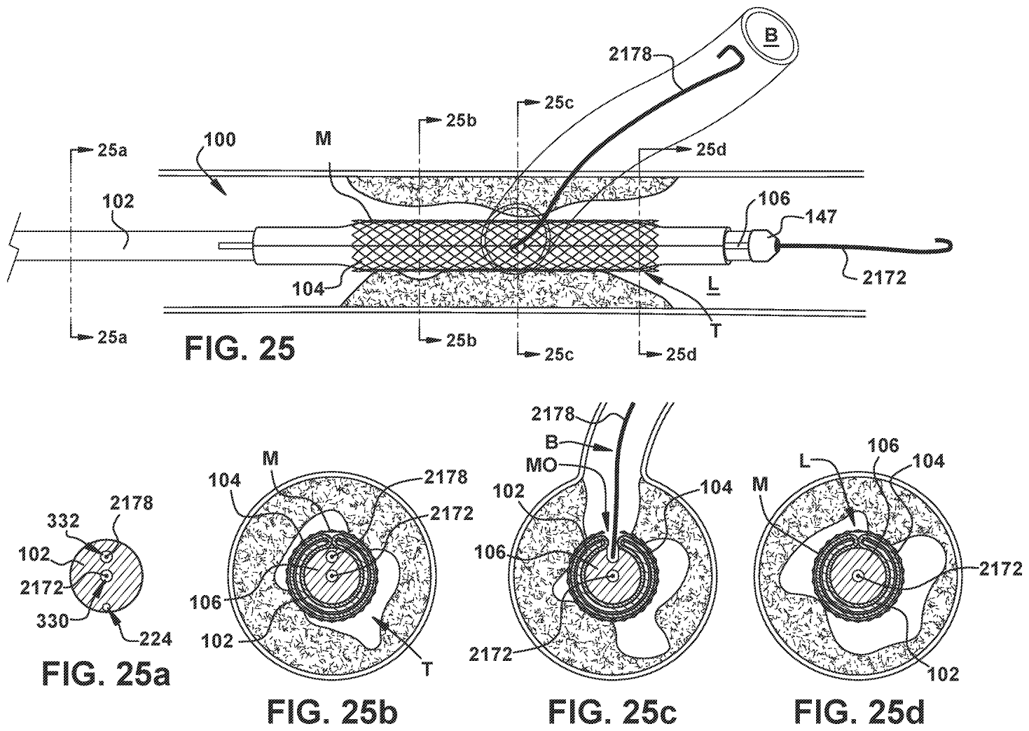

As shown in FIGS. 25-27, the balloon catheter 100 may have an expandable implant M, which can expand through external means (e.g., a balloon), mounted on a portion of the balloon catheter 100. In the example configuration shown in at least FIG. 25, the expandable implant M may be mounted circumferentially about at least a portion of the balloon 104.

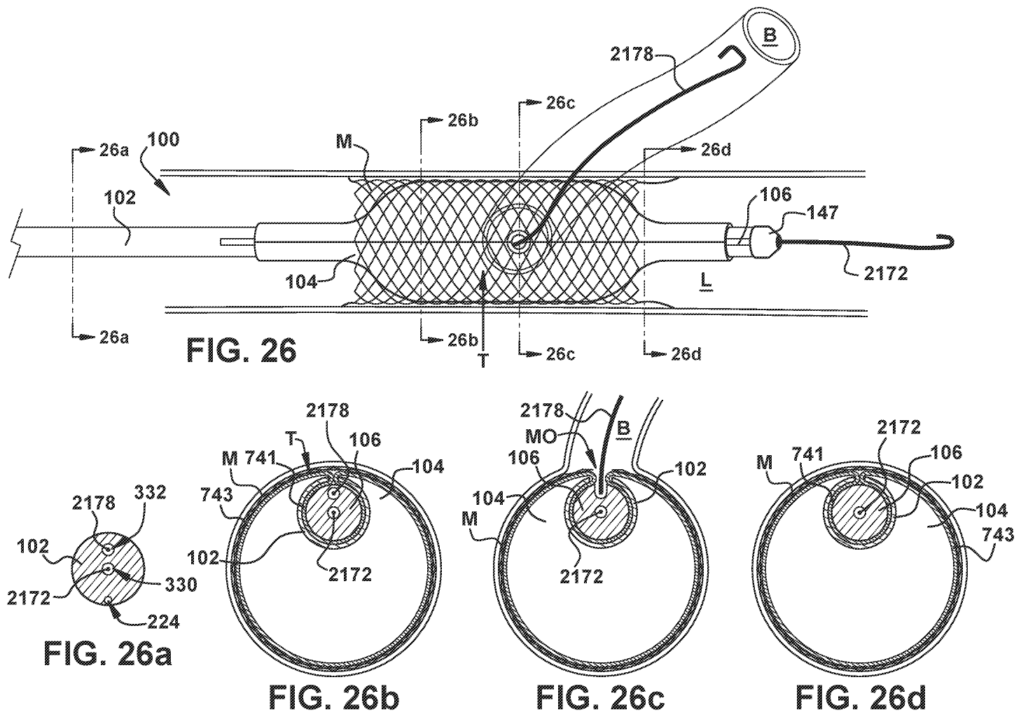

As shown in FIG. 25, the balloon catheter 100 with the mounted expandable implant M may be directed to the target patient tissue site T in a similar manner as described above. FIGS. 25a-d depict cross-sectional views taken at various locations along the balloon catheter 100, to show the arrangement of the balloon catheter 100, the expandable implant M, the first and second guidewires 2172, 2178, and the target patient tissue site Tin FIG. 25. As shown in FIG. 26, with the balloon catheter 100 at the target patient tissue site T, the balloon 104 is inflated to dilate the patient lumen L at the target patient tissue site T. Additionally, the inflating of the balloon 104 at least partially may help to cause the implant M to expand—i.e., to at least partially move toward the expanded condition. FIGS. 26a-d depict cross-sectional views taken at various locations along the balloon catheter 100, to show the arrangement of the balloon catheter 100, the expandable implant M, the first and second guidewires 2172, 2178, and the target patient tissue site T in FIG. 26.

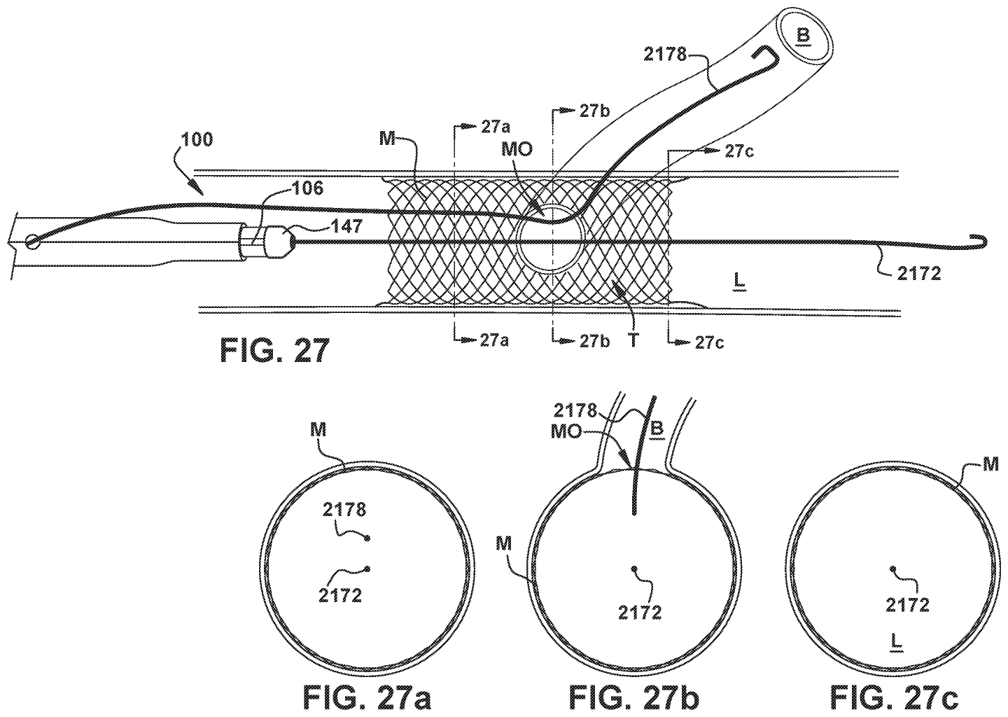

The balloon 104 is deflated after the patient lumen L at the target patient tissue site T has achieved a predetermined amount of dilation and after the expandable implant M has been expanded. As shown in FIG. 27, the balloon catheter 100 may then be removed from the target patient tissue site T, while the expanded implant M and the first and second guidewires 2172, 2178 are maintained substantially in position at the target patient site T. As shown in FIG. 27, the expandable implant M may have an implant sidewall opening MO that is positioned directed toward the patient lumen branch B and through which the second guidewire extends 2178. FIGS. 27a-c depict cross-sectional views taken at various locations along the expandable implant M, to show the arrangement of the expandable implant M, the first and second guidewires 2172, 2178, and the target patient tissue site T in FIG. 27. Because the first and second guidewires 2172, 2178 are maintained at the target patient tissue site T, one or more medical instruments may be advanced along one or both of the first and second guidewires 2172, 2178 to perform a medical procedure in the patient lumen L, in the patient lumen branch B, and/or at or adjacent to the target patient tissue site T.

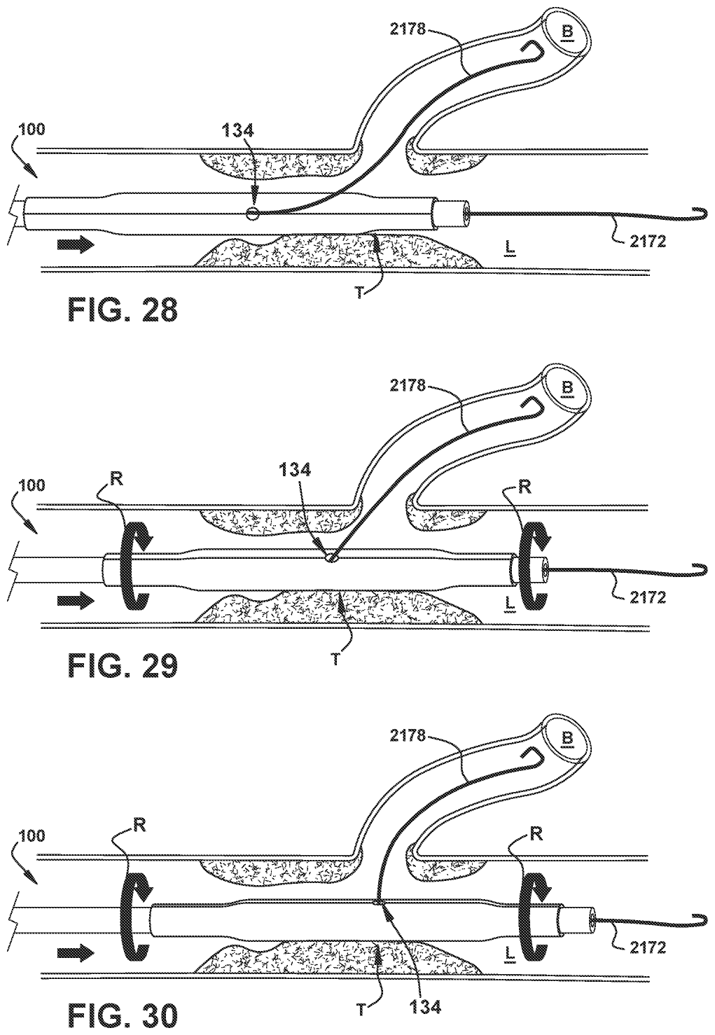

FIGS. 28-30 illustrate another example sequence of operation/use of the balloon catheter 100. As shown in FIG. 29, the balloon catheter 100 may be directed toward the target patient tissue site T with the balloon opening 134 being rotated away from the “side” of the patient lumen L that the patient lumen branch B extends from. In such case, as shown in FIG. 28-30, the second guidewire 2178 being in the patient lumen branch B helps circumferentially rotate (see the arrows “R” in FIGS. 29-30) the balloon catheter 100 as the balloon catheter 100 is directed to the target patient tissue site T along the first and second guidewires 2172, 2178 so that the balloon opening 134 is aligned with an opening of the patient lumen branch B once the balloon catheter 100 reaches the target patient tissue site T.

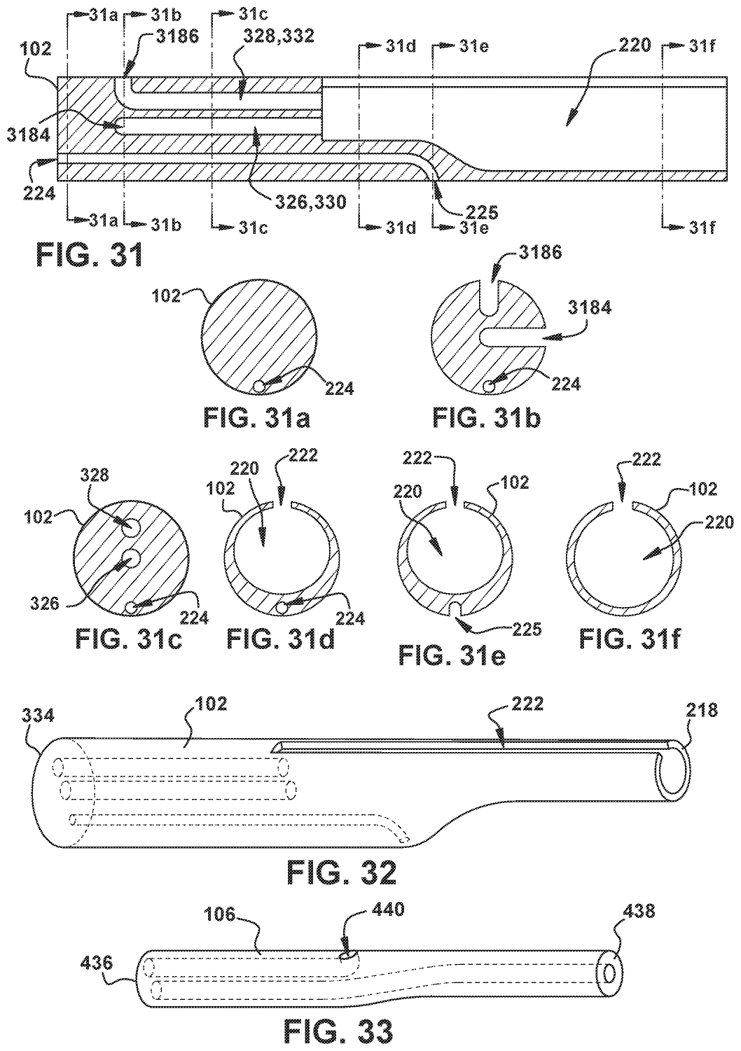

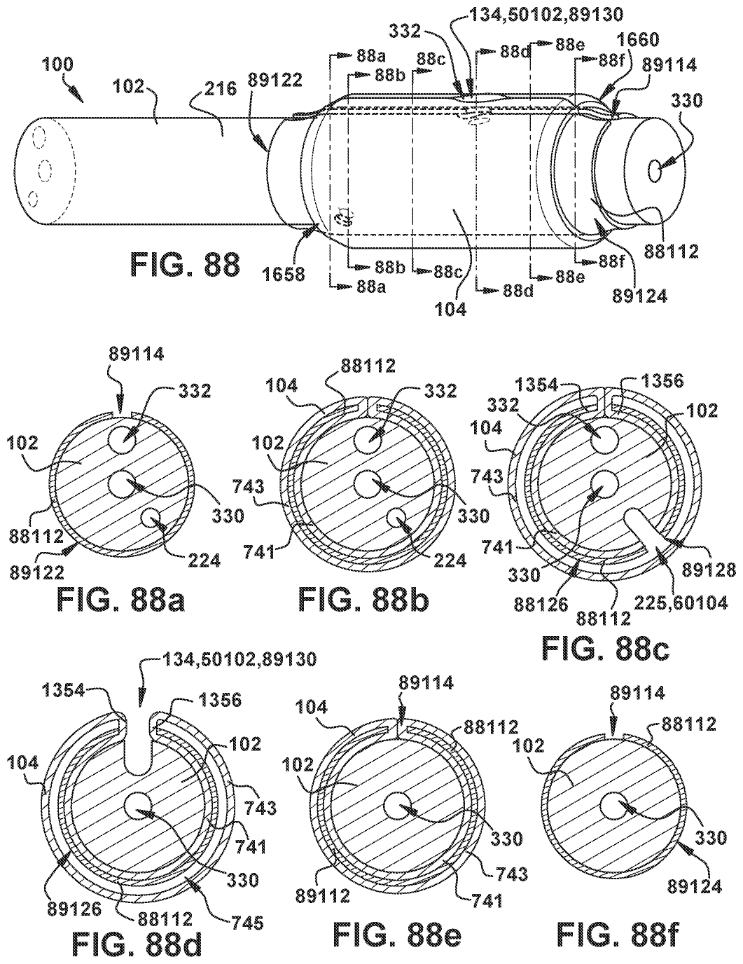

The above description and FIGS. 1-30 describe and show an example configuration of the individual pieces of the balloon catheter 100. The individual elements may have various other configurations and, thus, are not limited to those described above and shown in FIGS. 1-30. The below descriptions and FIGS. 31-87 described and show various other example configurations for the balloon catheter 100 and/or the individual elements of the balloon catheter 100. Description of common elements and operation similar to those previously described will not be repeated with respect to the configurations shown in FIGS. 31-87, but should instead be considered to be incorporated below by reference as appropriate. Furthermore, elements shown or described with respect to one example configuration may be shared by any of the other example configurations whether expressly stated, shown, or not.

FIG. 31 depicts another example configuration of the catheter 102. The catheter 102 of FIG. 31 is substantially similar to the catheter 102 of FIGS. 2-3, with some differences being described below. As shown in FIG. 31, the first portion 326 of the first balloon catheter lumen 330 extends substantially longitudinally from a first catheter sidewall opening 3184 to the distal catheter lumen 220, while the first portion 328 of the second balloon catheter lumen 332 extends substantially longitudinally from a second catheter sidewall opening 3186 to the distal catheter lumen 220. Accordingly, a balloon catheter 100 utilizing the catheter 102 of FIG. 31 has the first balloon catheter lumen 330 extending from the first catheter sidewall opening 3184 to the distal catheter tip 218, and the second balloon catheter lumen 332 extending from the second catheter sidewall opening 3186 to at least the fixer sidewall opening 440. FIGS. 31a-f depict cross-sectional views taken at various locations along the catheter 102 of FIG. 31.

Instead of each of the first portions 326, 328 of the first and second balloon catheter lumens 330, 332 extending from one of the first and second catheter sidewall openings 3184, 3186, only one of the first portions 326, 328 may extend from a corresponding one of the first and second catheter sidewall openings 3184, 3186 while the other of the first portions 326, 328 extends from the proximal catheter tip 334. In such a configuration, the catheter 102 may only include one of the first and second catheter sidewall openings 3184, 3186.

FIG. 32 depicts another example configuration of the catheter 102. The catheter 102 of FIG. 32 is substantially similar to the catheter 102 of FIGS. 2-3, with some differences being described below. As shown in FIG. 32, at least a portion of the catheter 102 inwardly tapers between the proximal and distal catheter tips 334, 218. The term “taper” is defined herein as a gradual diminution of thickness, diameter, or width in an elongated object, as is shown by the gradual diminution in diameter of a portion of the catheter 102 in FIG. 32. The term “inward” is defined herein as a taper that becomes gradually smaller, such as shown as the gradual diminution in diameter over a portion of the catheter 102 in FIG. 32.

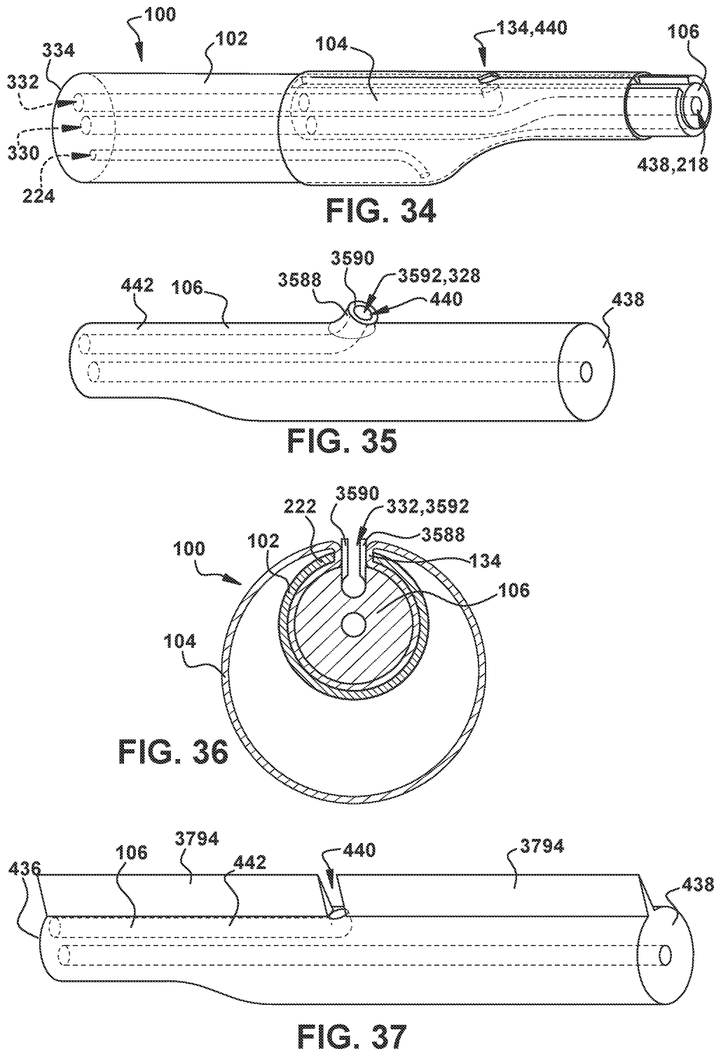

FIG. 33 depicts another example configuration of the fixer 106. The fixer 106 of FIG. 33 is substantially similar to the fixer 106 of FIGS. 4-5, with some differences being described below. As shown in FIG. 33, at least a portion of the fixer 106 inwardly tapers between the proximal and distal fixer tips 436, 438. FIG. 34 depicts a balloon catheter 100 that includes the catheter 102 and the fixer 106 of FIGS. 32-33. As shown in FIG. 34, the shape of the balloon 104 corresponds to the shape of at least the catheter 102 and, thus, at least a portion of the balloon 104 inwardly tapers along a longitudinal length of the balloon 104. Accordingly, at least a portion of the balloon catheter 100, as a whole, inwardly tapers along a longitudinal length of the balloon catheter 100. A balloon catheter 100 tapered in such a manner as shown in FIG. 34 may at least partially ease the insertion of the balloon catheter 100 to the target patient tissue site T.

FIGS. 35-36 depicts another example configuration of the fixer 106. The fixer 106 of FIGS. 35-36 is substantially similar to the fixer 106 of FIGS. 4-5, with some differences being described below. As shown in FIGS. 35-36, the fixer 106 includes a fixer branch 3588 extending from the fixer sidewall 442 adjacent the fixer sidewall opening 440. The fixer branch 3588 has an open branch tip 3590 and a branch lumen 3592 extending from the open branch tip 3590 to the fixer sidewall opening 440. As shown in FIG. 36, the fixer branch 3588 extends radially into at least one of the open catheter slit 222 and the balloon opening 134. The fixer branch 3588 may help a user locate the fixer sidewall opening 440 and/or the second balloon catheter lumen 332 more easily when the fixer sidewall opening 440 and/or the second balloon catheter lumen 332 are surrounded by the balloon material 1048.

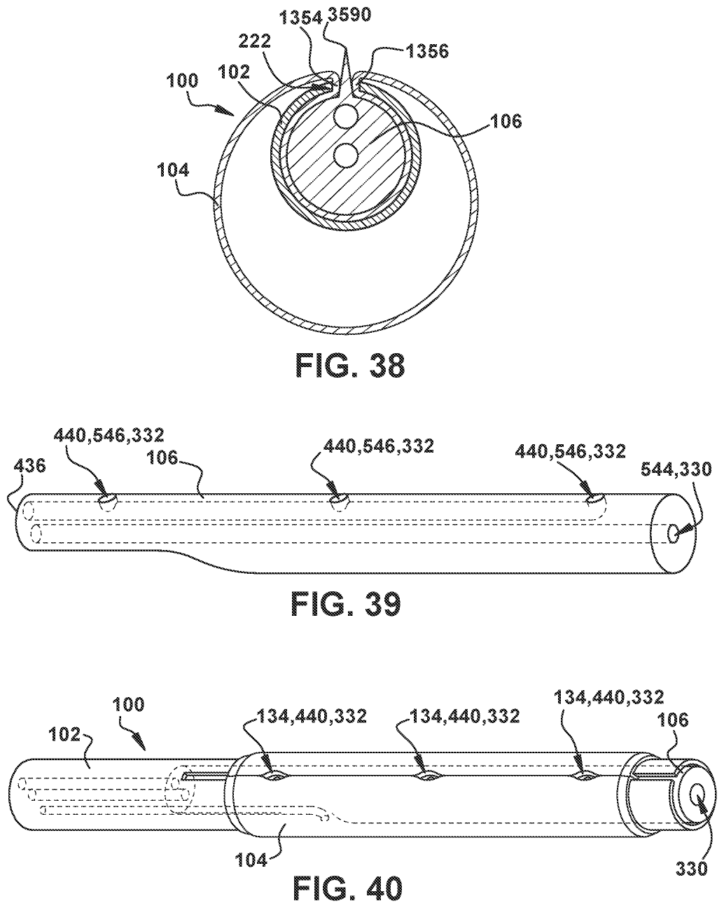

FIGS. 37-38 depicts another example configuration of the fixer 106. The fixer 106 of FIGS. 37-38 is substantially similar to the fixer 106 of FIGS. 4-5, with some differences being described below. As shown in FIGS. 37-38, the fixer 106 includes at least one fixer blade 3794 (as shown here, two fixer blades 3794) extending radially from the fixer sidewall 442. Each fixer blade 3794 may extend between the fixer sidewall opening 440 and one of the proximal and distal fixer tips 436, 438. As shown in FIG. 38, the fixer blades 3794 extend radially between adjacent circumferential end portions 1354, 1356 (i.e., the first and second balloon end portions 1354, 1356) of the at least one balloon 104 such that the adjacent circumferential end portions 1354, 1356 may be attached to the fixer blades 3794 during construction of the balloon catheter 100. The fixer blades 3794 may also extend radially into the open catheter slit 222. The fixer blade 3794 may be used to create a partial thickness linear cut in a diseased wall at the target patient tissue site T in order to help allow the balloon to expand at the target patient tissue site T.

FIG. 39 depicts another example configuration of the fixer 106. The fixer 106 of FIG. 39 is substantially similar to the fixer 106 of FIGS. 4-5, with some differences being described below. As shown in FIG. 39, the fixer 106 may have a plurality of fixer sidewall openings 440 (as shown here, three fixer sidewall openings 440). Each of the fixer sidewall openings 440 may be in fluid communication with the second portion 546 of the second balloon catheter lumen 332 such that the second balloon catheter lumen 332 may extend from the proximal catheter end portion 208 to at least each of the fixer sidewall openings 440. Alternatively, at least one of the fixer sidewall openings 440 may be in fluid communication with a balloon catheter lumen separate from the first and second balloon catheter lumens 330, 332.

FIG. 40 depicts another example configuration of the balloon catheter 100 that includes the fixer 106 of FIG. 39. As shown in FIG. 40, the balloon catheter 100 may be constructed such that the balloon 104 has a plurality of balloon openings 134 (as shown here, three balloon openings 134). The number of balloon openings 134 may be equal to the number of fixer sidewall openings 440. Each balloon opening 134 may be aligned with an associated fixer sidewall opening 440. Having more than one fixer/balloon opening 440/134 grants the user at least some positioning flexibility when positioning the balloon catheter 100 at the target patient tissue site T with at least one of the fixer/balloon openings 440/134 aligned with the patient lumen branch B.

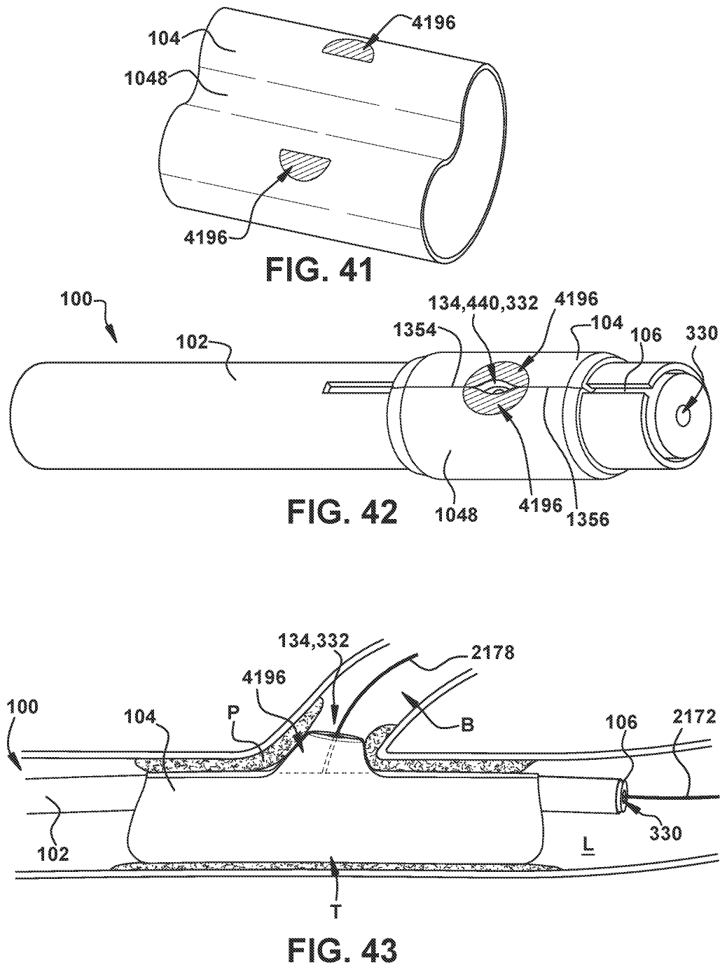

FIGS. 41-42 depict another example configuration of the balloon 104. The balloon 104 of FIGS. 41-42 is substantially similar to the balloon 104 of FIG. 1, with some differences being described below. As shown in FIG. 41, the balloon material 1048 of the balloon 104 includes a plurality of thin material portions 4196 (shown here as two thin material portions 4196) that are thinner in the radial direction than at least one other portion of the balloon material 1048. As shown in FIGS. 32, the thin material portions 4196 are at or adjacent the first and second balloon end portions 1354, 1356 so that when the balloon 104 is formed in the (or a similar) manner as described above, the thin material portions 4196 may be adjacent or directly adjacent one another. The thin material portions 4196 may also at least partially surround the balloon opening 134.

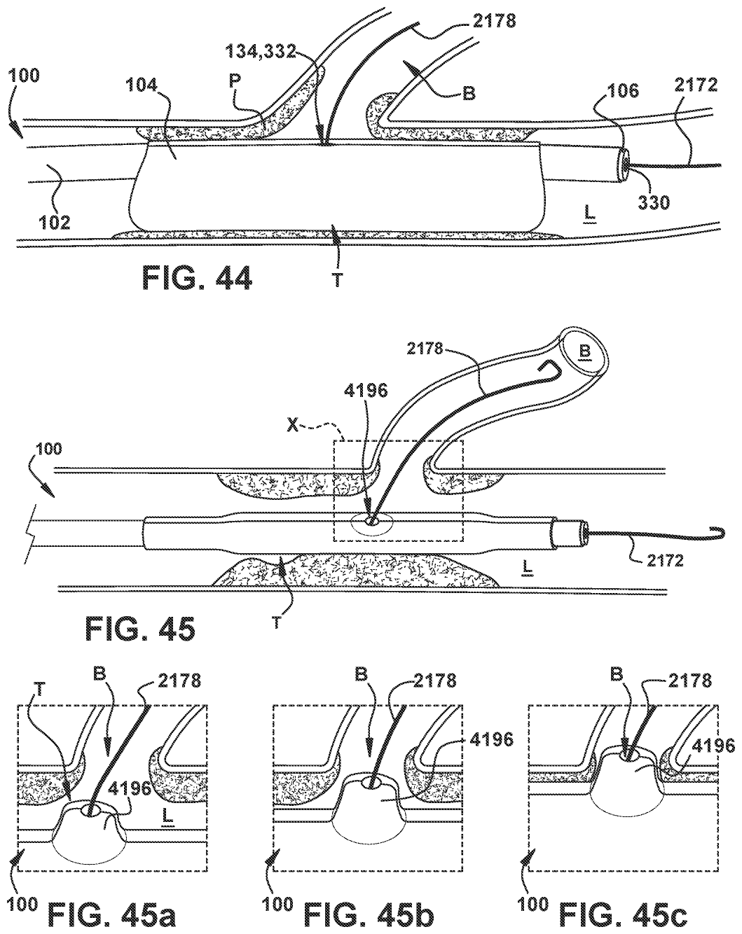

FIG. 43 depicts a balloon catheter 100 that includes the balloon 104 of FIGS. 41-42 inflated at the target patient tissue site T, while FIG. 44 depicts a balloon catheter 100 that includes the balloon 104 of FIG. 1 at the target patient tissue site T. The balloons 104 shown in FIGS. 43-44 may be inflated to the same or a similar fluid pressure. As shown in FIG. 43, the thin material portions 4196 of the balloon 104 expand more than the remaining portions of the balloon 104 during inflation of the balloon 104. Therefore, unlike the inflated balloon 104 in FIG. 44, the inflated balloon 104 in FIG. 43 protrudes into the patient lumen branch B because of the further inflated thin material portions 4196. The protrusion of the balloon 104 into the patient lumen branch B may at least partially help alleviate a stenosis and/or an occlusion in the patient lumen branch B. The protruding portion 4196 of the balloon 104 may also at least partially help prevent displaceable materials (e.g., plaque P) in the patient lumen L from being displaced during the dilation of the patient lumen L at the target patient tissue site T into the patient lumen branch B.

FIG. 45 also depicts the balloon catheter 100 of FIG. 42 at a target patient tissue site T, while FIGS. 45a-c illustrate an example sequence of operation of a portion X of the balloon catheter 100 from FIG. 45. As shown in FIGS. 45a-c, the thin material portions 4196 of the balloon 104 expand around and along the second guidewire 2178 and helps align the balloon catheter 100 at the target patient tissue site T. In particular, the expansion of the thin material portions 4196 around and along the second guidewire 2178 at least partially helps urge the balloon opening 134 toward the patient lumen branch B so that the balloon opening 134 (and, e.g., at the thin material portions 4196 of the balloon 104) rests in or adjacent to the origin of the patient lumen branch B when the balloon is inflated.

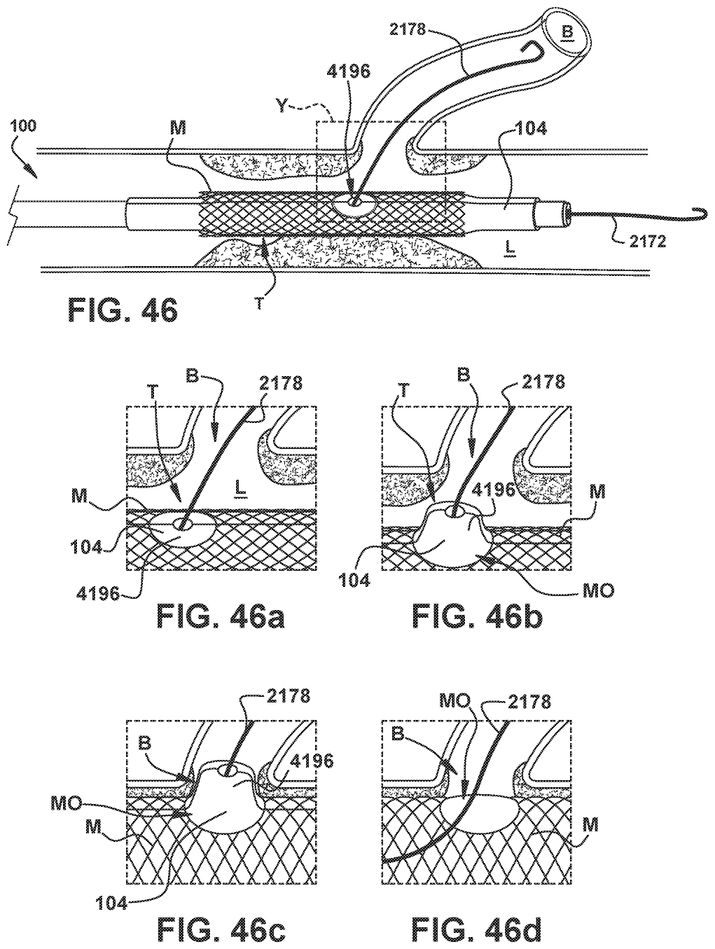

FIG. 46 depicts the balloon catheter 100 of FIG. 42 at a target patient tissue site T, while FIGS. 46a-d illustrate an example sequence of operation of a portion Y of the balloon catheter 100 from FIG. 46. FIGS. 46-46c also includes the expandable implant M may be mounted circumferentially about at least a portion of the balloon 104. As shown in FIGS. 46a-c, in addition to helping align the balloon catheter 100 at the target patient tissue site, the expansion of the thin material portions 4196 of the balloon 104 expand around the second guidewire 2178 and helps align the expandable implant M at the target patient tissue site T. In particular, the expansion of the thin material portions 4196 around the second guidewire 2178 at least partially helps urge the balloon opening 134 and the implant sidewall opening MO toward the patient lumen branch B so that the balloon opening 134 rests at the origin of the patient lumen branch B and the implant sidewall opening MO is aligned with the ostium of the patient lumen branch B when the balloon 104 is inflated and the expandable implant M is expanded.

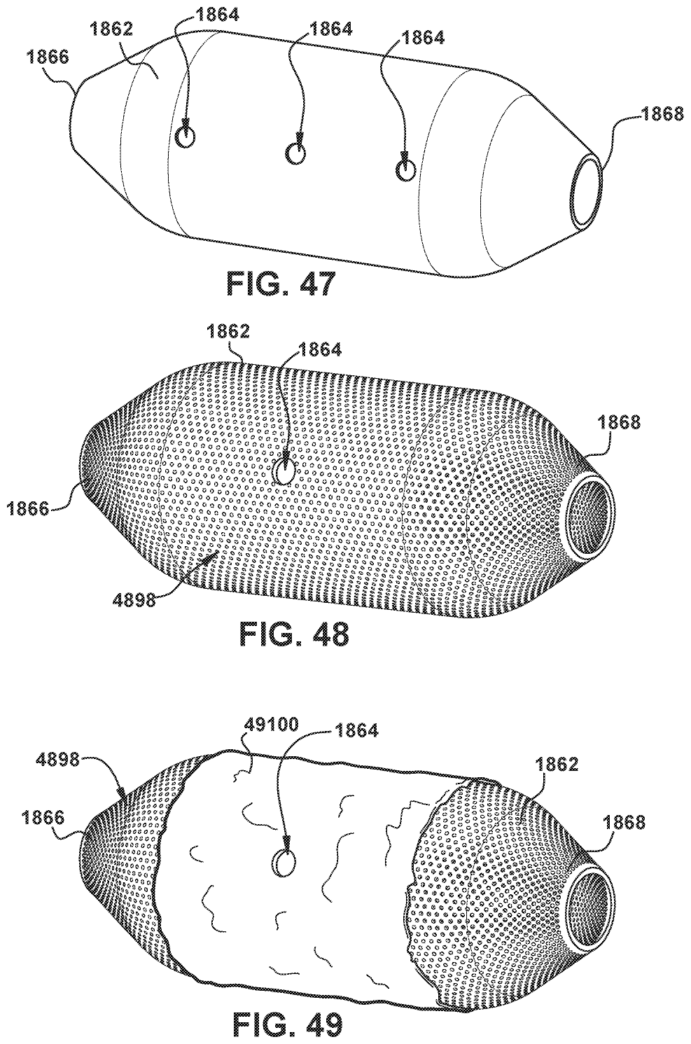

FIG. 47 depicts another example configuration of the wrap 1862. The wrap 1862 of FIG. 47 is substantially similar to the wrap 1862 of FIGS. 18-20c, with some differences being described below. As shown in FIG. 47, the wrap 1862 may have a plurality of the wrap openings 1864 extending radially therethrough. Each wrap opening 1864 may be aligned with an associated fixer sidewall opening 440 and an associated balloon opening 134.

FIG. 48 depicts another example configuration of the wrap 1862. The wrap 1862 of FIG. 48 is substantially similar to the wrap 1862 of FIGS. 18-20c, with some differences being described below. As shown in FIG. 48, the wrap 1862 may include a plurality of perforations 4898. In use, a therapeutic substance may be embedded within the outer surface of the balloon 104 or held wedged between the balloon 104 and the wrap 1862. When the balloon 104 is in collapsed condition during insertion, the therapeutic substance may be at least partially protected and not exposed to bodily fluids (e.g., flowing blood) that could wash away the therapeutic substance prior to delivery at the target patient tissue site T. At the target patient tissue site T, the balloon 104 is inflated and the wrap 1862 is expanded. As the wrap expands 1862 the perforations 4898 become visible and open up. Through these open perforations 4898, the therapeutic substance elutes to structures/walls at the target patient tissue site T.

FIG. 49 depicts another example configuration of the wrap 1862. The wrap 1862 of FIG. 49 is substantially similar to the wrap 1862 of FIG. 48 with perforations 4898 or the wrap 1862 of FIGS. 18-20c without perforations, with some differences being described below. As shown in FIG. 49, the wrap 1862 may carry at least one therapeutic substance 49100. For example, the therapeutic substance 49100 may coat the wrap 1862, may be embedded in the wrap 1862, and/or may be carried on the wrap 1862 via one or more holding mechanisms. The wrap 1862 carrying the therapeutic substance 49100 may be useful for delivering the therapeutic substance 49100 to at least the target patient tissue site T during use of the balloon catheter 100. While the wrap 1862 of FIG. 49 is shown as having the perforations 4898, the wrap 1962 may be constructed such that the perforations 4898 are omitted.

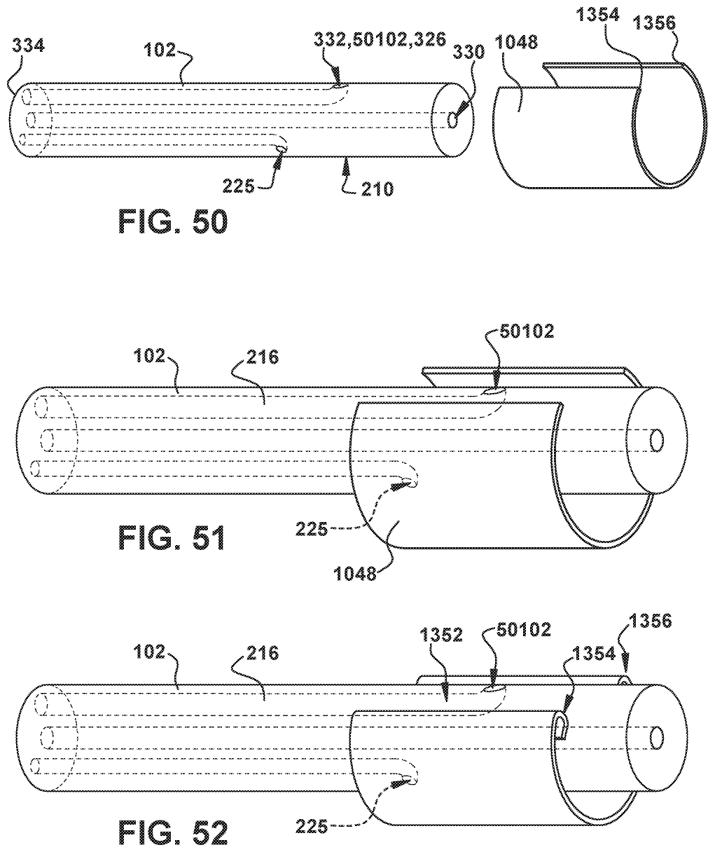

FIGS. 50-56 depict an example sequence for construction another example configuration of the balloon catheter 100. The balloon catheter 100 constructed with the process shown in FIGS. 50-56 is substantially similar to the balloon catheter 100 of FIG. 1, with some differences being described below. As shown in FIG. 50, to construct the balloon catheter 100 in FIGS. 50-56, the catheter 102 is provided. Unlike the catheter 102 of FIGS. 2-3, the catheter 102 of FIG. 50 does not include the distal catheter lumen 220 and, thus, the balloon catheter 100 constructed in the process shown in FIGS. 50-56 does not include a fixer 106 that is inserted into a distal catheter lumen 220. The second balloon catheter lumen 332 of FIG. 50 extends substantially longitudinally through the catheter 102 from the proximal catheter tip 334 to a distal catheter sidewall opening 50102 at the distal catheter end portion 210.

As shown in FIG. 50, a balloon material 1048 is also provided. Unlike the balloon material 1048 of FIG. 10 which has an elliptical shape in radial cross-section, the balloon material 1048 of FIG. 50 is a sheet that is manipulated into an arcuate shape in radial cross-section. As shown in FIG. 51, the arcuate balloon material 1048 may be placed at least partially circumferentially about the outer catheter surface 216 so that the balloon material 1048 overlies the catheter inflation opening 225. As shown in FIG. 52, the balloon material 1048 may form an open balloon slit 1352 that is aligned with the distal catheter sidewall opening 50102. The balloon material 1048 defines first and second balloon end portions 1354, 1356 on opposing sides of the open balloon slit 1352. The first and second balloon end portions 1354, 1356 are then folded inward (i.e., downwards and toward the catheter 102 in the orientation of FIG. 52) over the outer catheter surface 216.

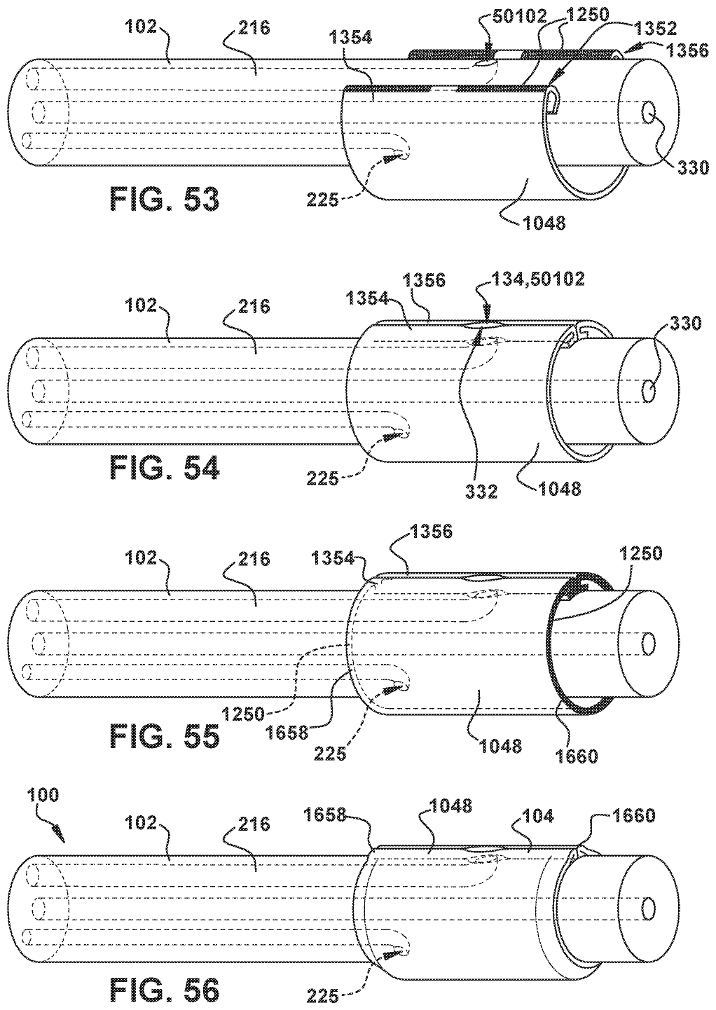

As shown in FIG. 53, an adhesive 1250 may be applied to at least one of the balloon material 1048 and the outer catheter surface 216 on or adjacent to the first and second balloon end portions 1354, 1356. As shown in FIGS. 53-54, via the adhesive 1250, a portion of the first balloon end portion 1354 (e.g., the folded portion of the first balloon end portion 1354) is attached to the outer catheter surface 216 and a portion the second balloon end portion 1356 (e.g., the folded portion of the second balloon end portion 1356) in such a manner that the balloon opening 134 is defined between the first and second balloon end portions 1354, 1356. The second balloon end portion 1356 (e.g., the folded portion of the second balloon end portion 1356) may also be attached to the outer catheter surface 216 via the adhesive 1250. When attached as described above, the at least one balloon opening 134 is aligned with the distal catheter sidewall opening 50102 such that the second balloon catheter lumen 332 now extends from the proximal catheter tip 334 to the balloon opening 134.

As shown in FIGS. 55-56, at least a portion of the proximal balloon material end 1658 is circumferentially attached to at least a portion of the outer catheter surface 216 via an adhesive 1250. At least a portion of the distal balloon material end 1660 is also circumferentially attached to at least a portion of the outer catheter surface 216 via an adhesive 1250. The expandable balloon 104 is formed having the balloon opening 134 once the balloon material 1048 is attached as described above.

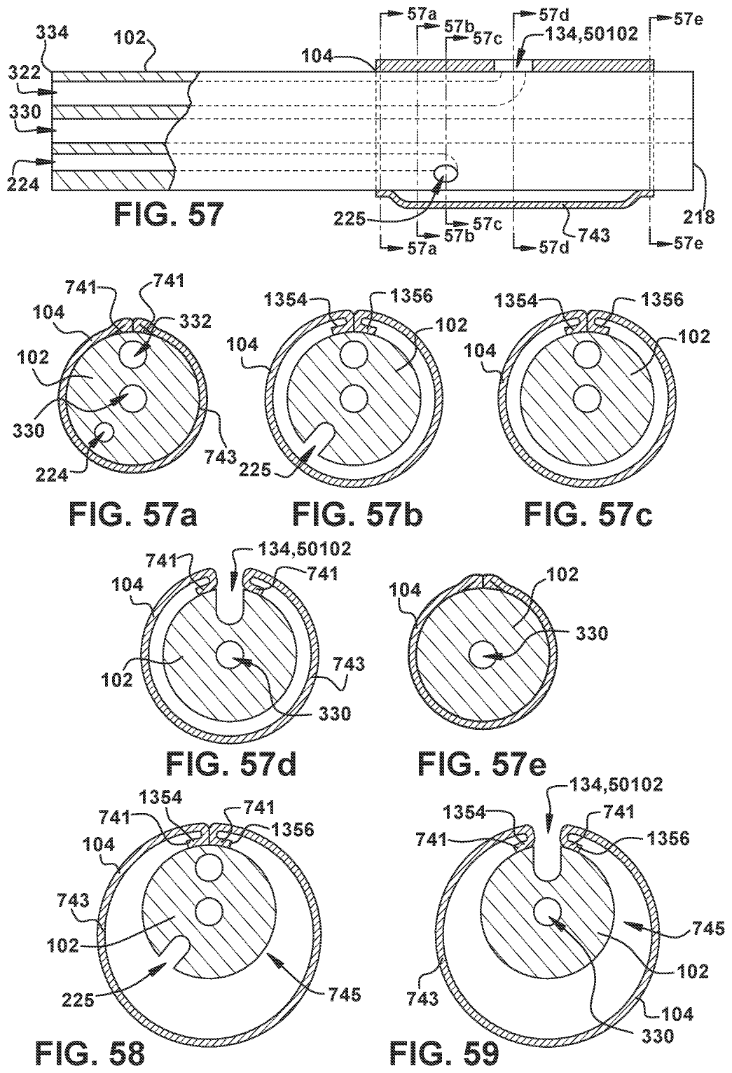

FIG. 57 schematically illustrates the balloon catheter 100 constructed as shown in FIGS. 50-56. As shown in FIG. 57, the first balloon catheter lumen 332 extends from the proximal catheter tip 334 to the distal catheter tip 218, while the second balloon catheter lumen 332 now extends from the proximal catheter tip 334 to the balloon opening 134. FIGS. 57a-e depict cross-sectional views taken at various locations along the balloon catheter 100 of FIG. 57, to show the arrangement of the catheter 102 and the uninflated balloon 104. FIGS. 58 and 59 depict cross-sectional views of FIG. 57b and FIG. 57d, respectively, after the balloon 104 has been at least partially inflated. The balloon 104 may be configured such that only the outer balloon portion 743 moves during inflation of the balloon 104 because the inner balloon portion 741 is fixed to the catheter 102 via the folded first and second balloon end portions 1354, 1356.

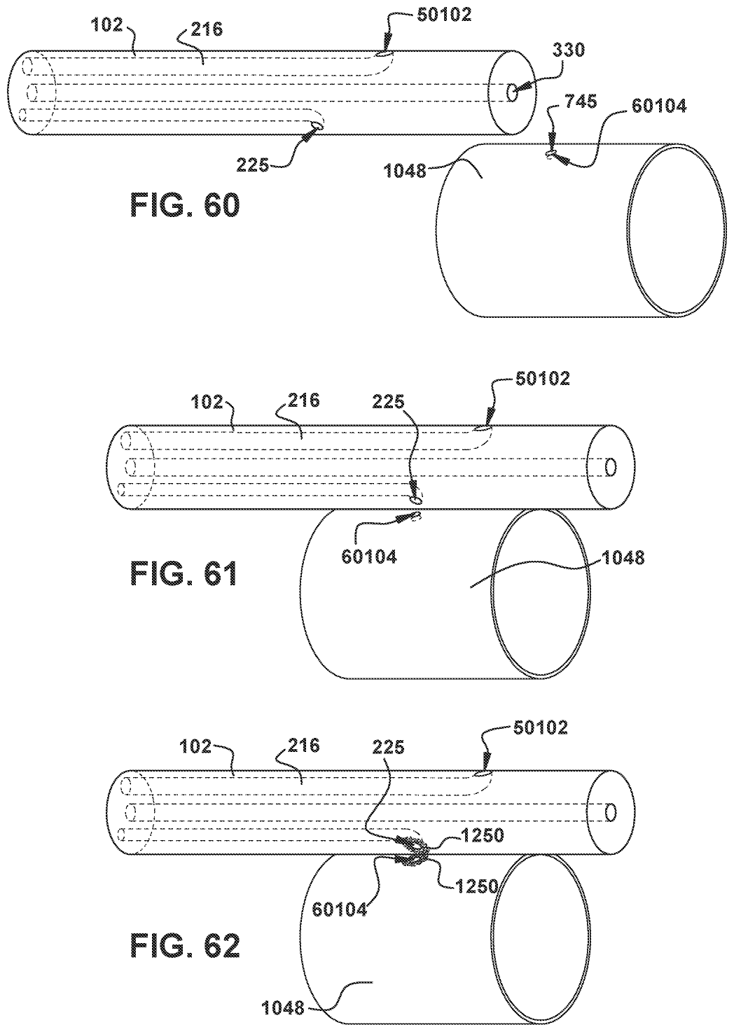

FIGS. 60-69 depict an example sequence for construction another example configuration of the balloon catheter 100. The balloon catheter 100 constructed with the process shown in FIGS. 60-69 is substantially similar to the balloon catheter 100 of FIG. 57, with some differences being described below. As shown in FIG. 60, to construct the balloon catheter 100 in FIGS. 60-69, the catheter 102 is provided. The catheter 102 of FIG. 60 is the same as or substantially similar to the catheter 102 of FIG. 50.

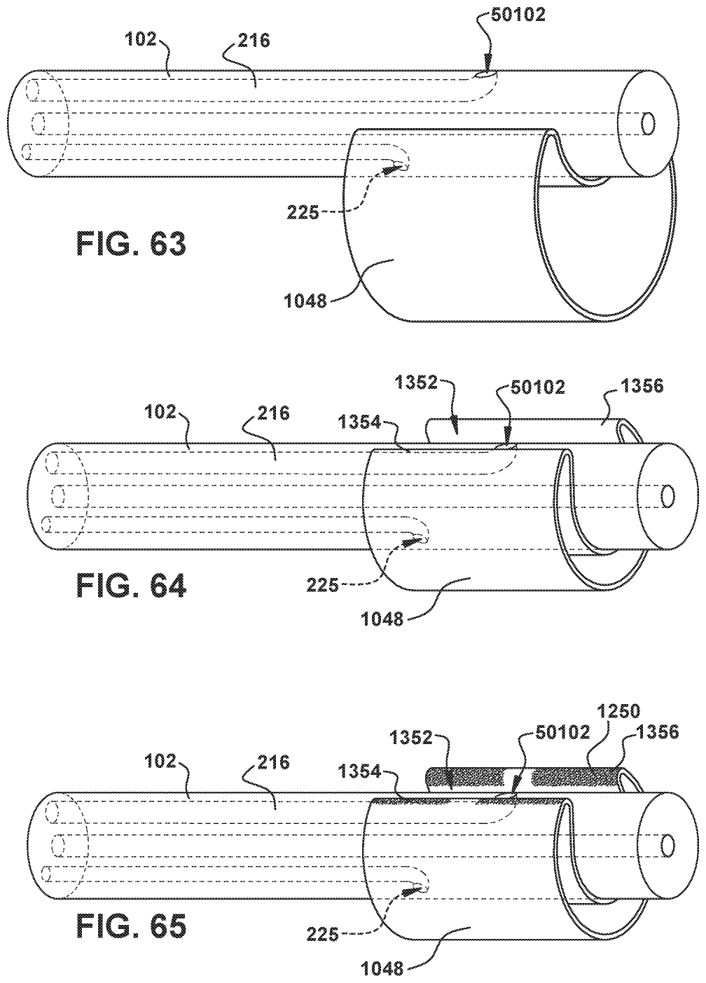

As shown in FIG. 60, a balloon material 1048 is also provided. The balloon material 1048 of FIG. 60, like the balloon material 1048 of FIG. 10, has an elliptical shape in radial cross-section. However, unlike the balloon material 1048 of FIG. 10, the balloon material 1048 of FIG. 60 is formed as an expandable balloon 104 having a balloon inflation opening 60104 that is in fluid communication with the inflatable volume 745 of the fully formed balloon 104. As shown in FIG. 61, the balloon material 1048 may be aligned with the catheter 102 such that the balloon inflation opening 60104 is aligned with the catheter inflation opening 225. As shown in FIG. 52 an adhesive 1250 may be applied to the catheter 102 and/or the balloon material 1048 adjacent the balloon inflation opening 60104 and/or the catheter inflation opening 225. As shown in FIGS. 62-63, via the adhesive 1250, a portion of the balloon material 1048 may be attached to the outer catheter surface 216 so that the balloon inflation opening 60104 is in fluid communication with the catheter inflation opening 225, and also the inflation lumen 224 is in fluid communication with the inflatable volume 745 when the balloon 104 is fully formed.

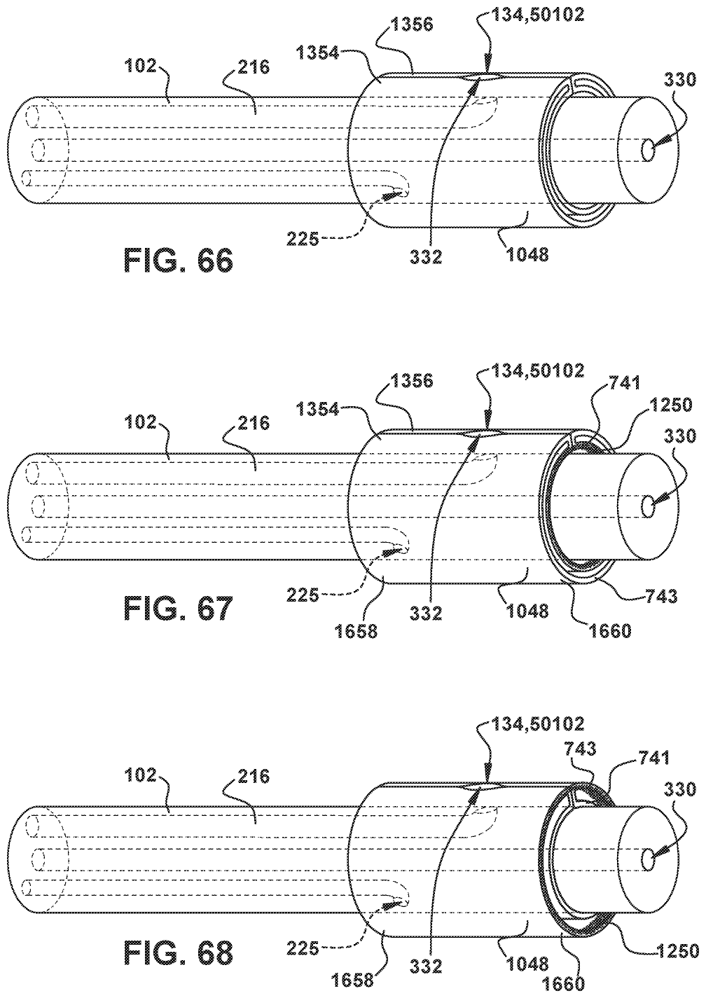

As shown in FIGS. 63-64, the balloon material 1048 is wrapped at least partially circumferentially about the outer catheter surface 216. As shown in FIG. 64, the balloon material 1048 may form an open balloon slit 1352 that is aligned with the distal catheter sidewall opening 50102. The balloon material 1048 defines first and second balloon end portions 1354, 1356 on opposing sides of the open balloon slit 1352. As shown in FIG. 65, an adhesive 1250 may be applied to at least one of the balloon material 1048 and the outer catheter surface 216 on or adjacent to the first and second balloon end portions 1354, 1356. As shown in FIGS. 65-66, via the adhesive 1250, a portion of the first balloon end portion 1354 is attached to the outer catheter surface 216 and a portion the second balloon end portion 1356 in such a manner that the balloon opening 134 is defined between the first and second balloon end portions 1354, 1356. The second balloon end portion 1356 may also be attached to the outer catheter surface 216 via the adhesive 1250. When attached as described above, the at least one balloon opening 134 is aligned with the distal catheter sidewall opening 50102 such that the second balloon catheter lumen 332 now extends from the proximal catheter tip 334 to the balloon opening 134.

At least a portion of an inner balloon portion 741 at the proximal balloon material end 1658 is circumferentially attached to at least a portion of the outer catheter surface 216 via an adhesive 1250. As shown in FIG. 67, at least a portion of the inner balloon portion 741 at the distal balloon material end 1660 is also circumferentially attached to at least a portion of the outer catheter surface 216 via an adhesive 1250. At least a portion of an outer balloon portion 743 at the proximal balloon material end 1658 is circumferentially attached to at least a portion of at least one of the inner balloon portion 741 and the outer catheter surface 216 via an adhesive 1250. As shown in FIG. 68, at least a portion of the outer balloon portion 743 at the distal balloon material end 1660 is also circumferentially attached to at least a portion of at least one of the inner balloon portion 741 and the outer catheter surface 216 via an adhesive 1250 to form the inflatable volume 745.

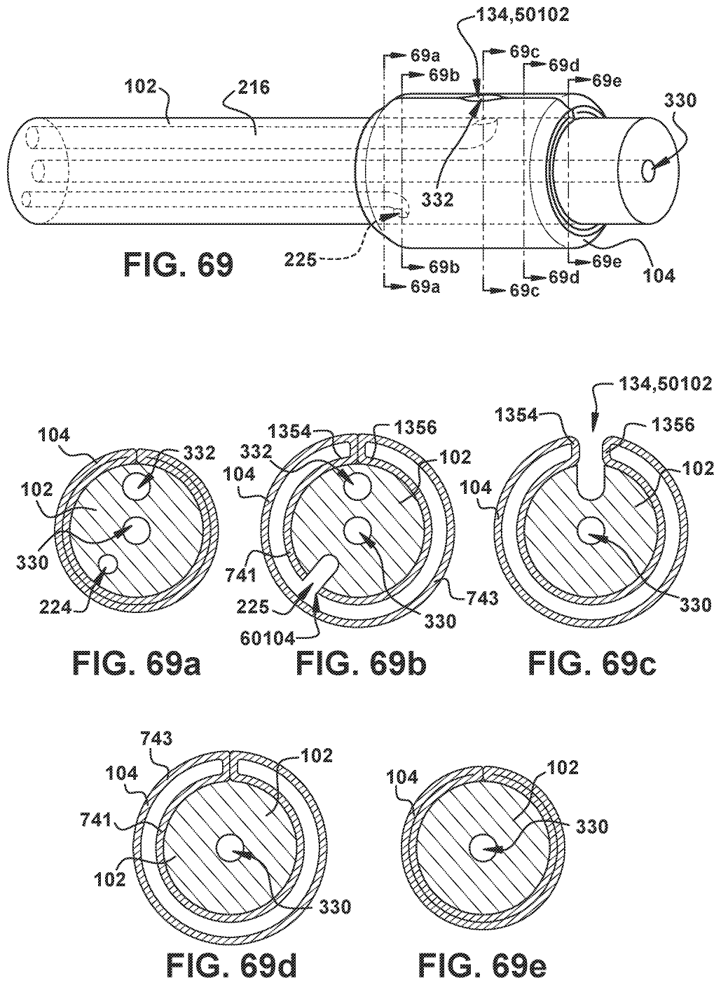

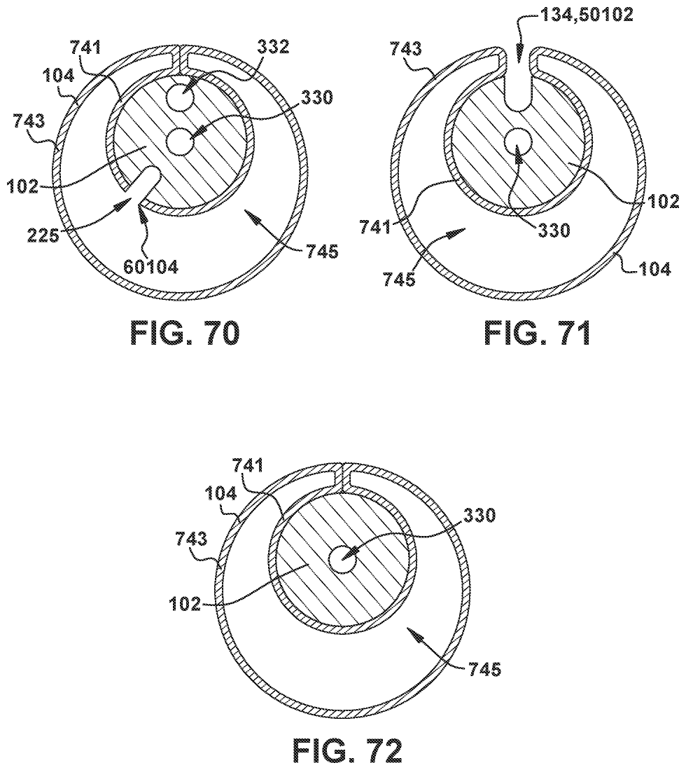

As shown in FIG. 69, the expandable balloon 104 is formed having the balloon opening 134 once the balloon material 1048 is attached as described above. FIGS. 69a-e depict cross-sectional views taken at various locations along the balloon catheter 100 of FIG. 69, to show the arrangement of the catheter 102 and the uninflated balloon 104. FIGS. 70-72 depict cross-sectional views of FIGS. 69b-d, respectively, after the balloon 104 has been at least partially inflated. The balloon 104 may be configured such that only the outer balloon portion 743 moves during inflation of the balloon 104 because the inner balloon portion 741 is fixed to the catheter 102.

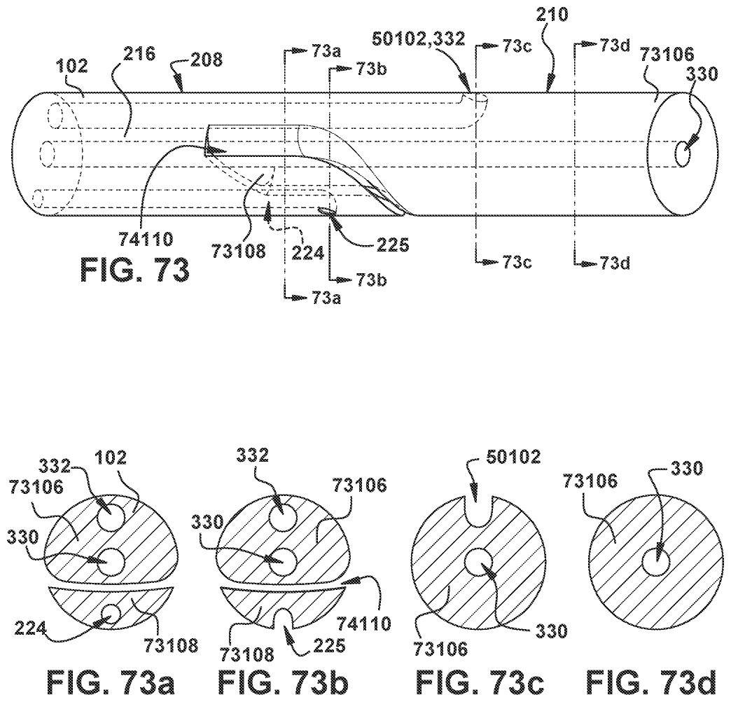

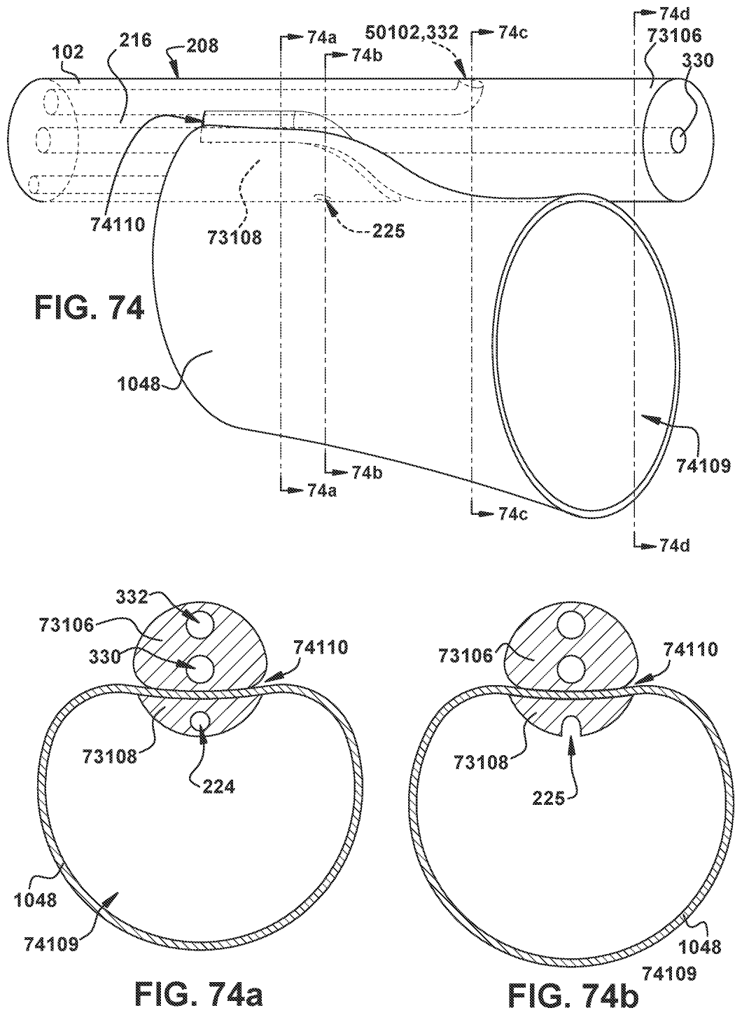

FIGS. 73-81 depict an example sequence for construction another example configuration of the balloon catheter 100. The balloon catheter 100 constructed with the process shown in FIGS. 73-81 is substantially similar to the balloon catheter 100 of FIG. 57, with some differences being described below. As shown in FIG. 73, to construct the balloon catheter 100 in FIGS. 73-81, the catheter 102 is provided. The catheter 102 of FIG. 73 is similar to the catheter 102 of FIG. 57. However, unlike the catheter 102 of FIG. 57, the distal catheter end portion 210 of the catheter 102 of FIG. 73 includes a primary catheter branch 73106 and a secondary catheter branch 73108 extending longitudinally from the proximal catheter end portion 208. The primary catheter branch 73106 includes the first and second balloon catheter lumens 330, 332 and the distal catheter sidewall opening 50102, while the secondary catheter branch 73108 includes the inflation lumen 224 and the catheter inflation opening 225. FIGS. 73a-d depict cross-sectional views taken at various locations along the catheter 102.

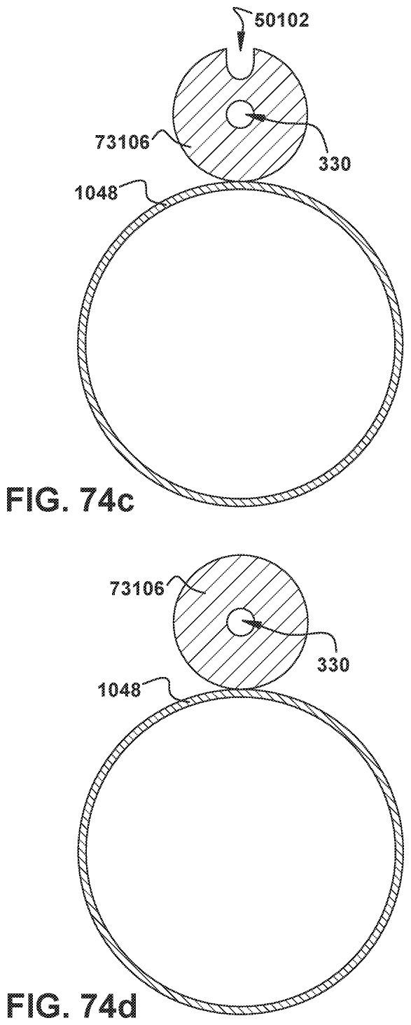

As shown in FIG. 74, a balloon material 1048 is also provided. The balloon material 1048 of FIG. 74 is the same as or substantially similar to the balloon material 1048 of FIG. 1. As shown in FIG. 74, the balloon material 1048 is inserted over the secondary catheter branch 73108 so that the secondary catheter branch 73108 is positioned in an interior 74109 of the balloon material 1048 and a portion of the balloon material 1048 is positioned in a space 74110 between the primary and secondary catheter branches 73106, 73108. FIGS. 74a-d depict cross-sectional views taken at various locations along the catheter 102 and the balloon material 1048, to show the arrangement of the catheter 102 and the balloon material 1048 in FIG. 74.

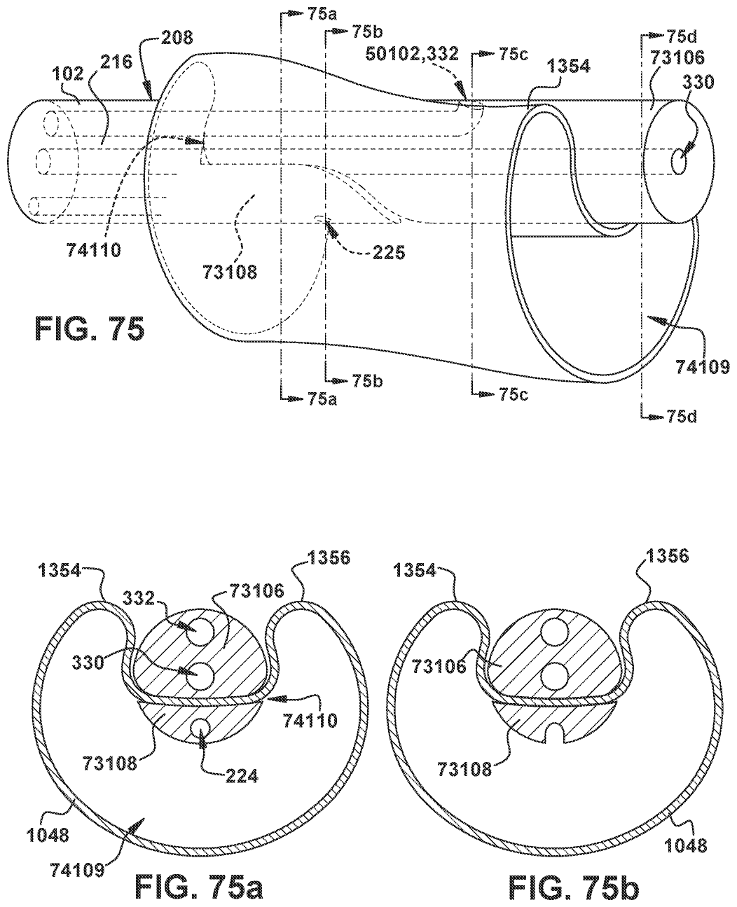

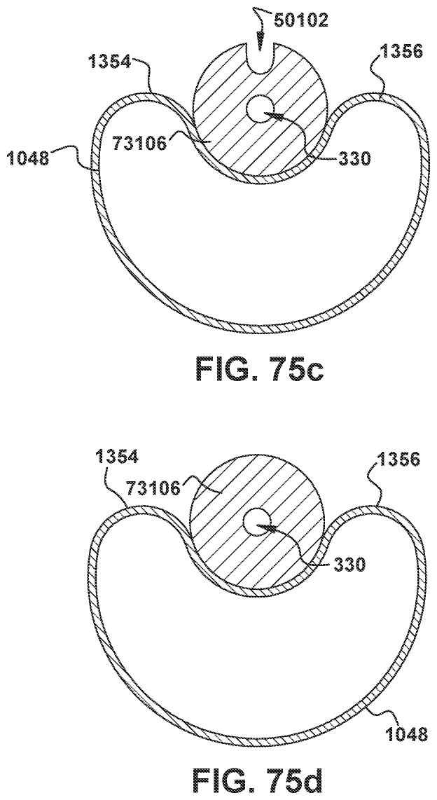

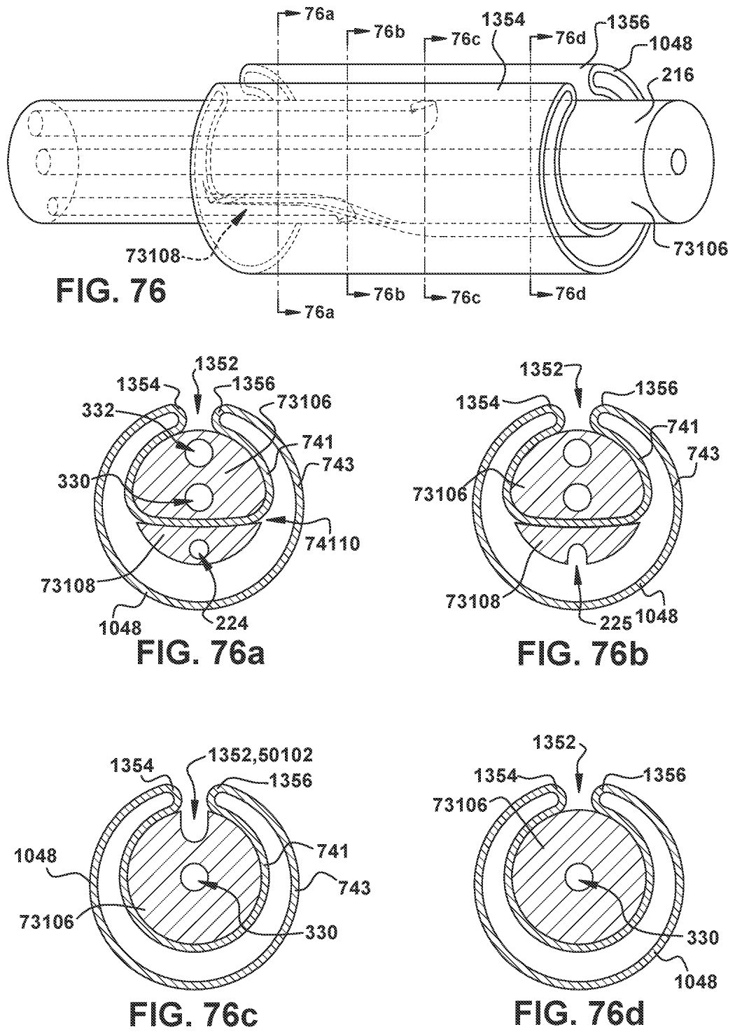

As shown in FIGS. 75-76, a first balloon end portion 1354 is wrapped at least partially circumferentially about the outer catheter surface 216 and positioned adjacent a second balloon end portion 1356 so that an open balloon slit 1352 is formed between the first and second balloon end portions 1354, 1356 and the open balloon slit 1352 is aligned with the distal catheter sidewall opening 50102. FIGS. 75a-d depict cross-sectional views taken at various locations along the catheter 102 and the balloon material 1048, to show the arrangement of the catheter 102 and the balloon material 1048 in FIG. 75. FIGS. 76a-d depict cross-sectional views taken at various locations along the catheter 102 and the balloon material 1048, to show the arrangement of the catheter 102 and the balloon material 1048 in FIG. 76.

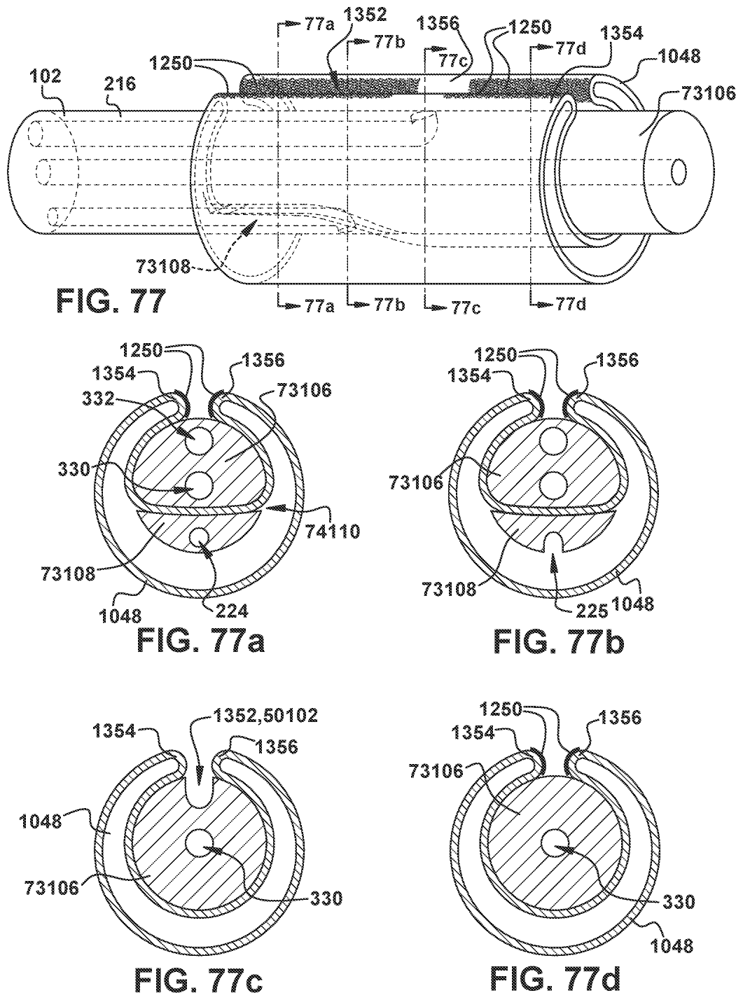

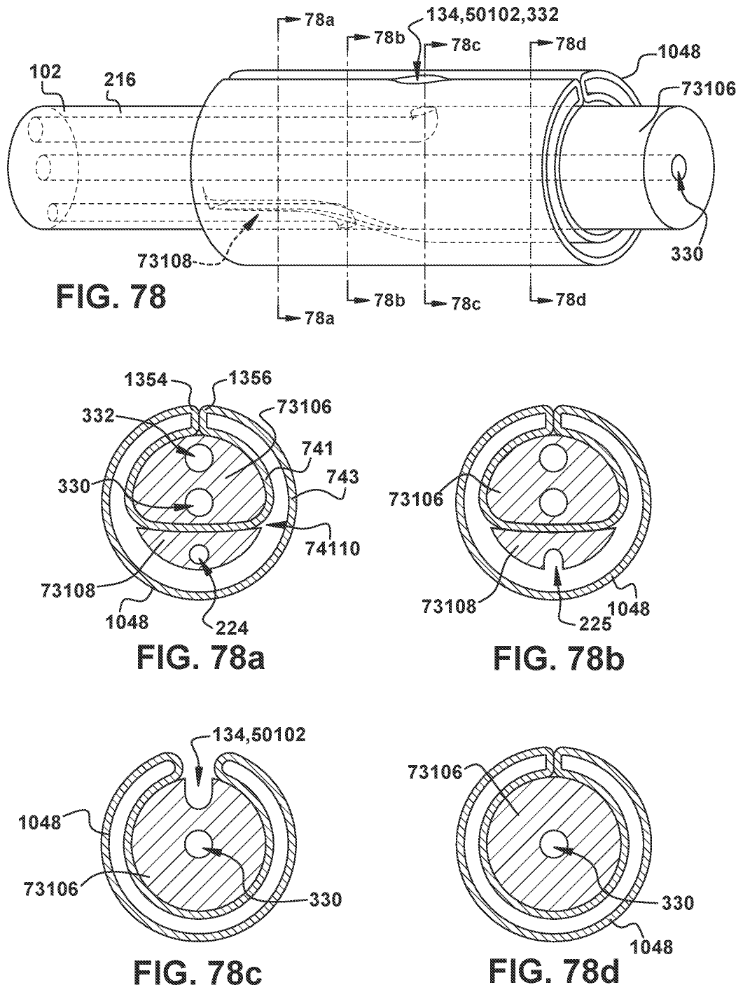

As shown in FIG. 77, an adhesive 1250 may be applied to at least one of the balloon material 1048 and the outer catheter surface 216 on or adjacent to the first and second balloon end portions 1354, 1356. FIGS. 77a-d depict cross-sectional views taken at various locations along the catheter 102 and the balloon material 1048, to show the arrangement of the catheter 102 and the balloon material 1048 in FIG. 77. As shown in FIG. 78, via the adhesive 1250, a portion of the first balloon end portion 1354 is attached to the outer catheter surface 216 and a portion the second balloon end portion 1356 in such a manner that the balloon opening 134 is defined between the first and second balloon end portions 1354, 1356. The second balloon end portion 1356 may also be attached to the outer catheter surface 216 via the adhesive 1250. When attached as described above, the at least one balloon opening 134 is aligned with the distal catheter sidewall opening 50102 such that the second balloon catheter lumen 332 now extends from the proximal catheter tip 334 to the balloon opening 134. FIGS. 78a-d depict cross-sectional views taken at various locations along the catheter 102 and the balloon material 1048, to show the arrangement of the catheter 102 and the balloon material 1048 in FIG. 78.

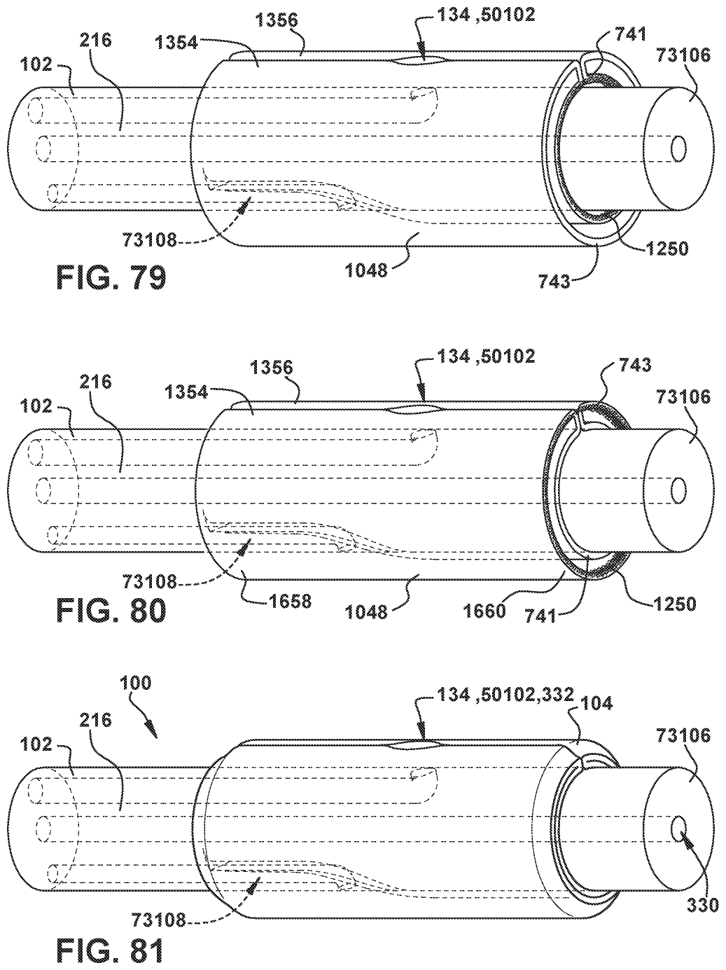

At least a portion of an inner balloon portion 741 at the proximal balloon material end 1658 is circumferentially attached to at least a portion of the outer catheter surface 216 (such as to, e.g., the outer catheter surface 216 at least at the primary and/or the secondary catheter branches 73106, 73108) via an adhesive 1250. As shown in FIG. 79, at least a portion of the inner balloon portion 741 at the distal balloon material end 1660 is also circumferentially attached to at least a portion of the outer catheter surface 216 via an adhesive 1250. At least a portion of an outer balloon portion 743 at the proximal balloon material end 1658 is circumferentially attached to at least a portion of at least one of the inner balloon portion 741 and the outer catheter surface 216 (such as to, e.g., the outer catheter surface 216 at least at the primary and/or the secondary catheter branches 73106, 73108) via an adhesive 1250. As shown in FIGS. 80-81, at least a portion of the outer balloon portion 743 at the distal balloon material end 1660 is also circumferentially attached to at least a portion of at least one of the inner balloon portion 741 and the outer catheter surface 216 via an adhesive 1250 to form the inflatable volume 745. The expandable balloon 104 is formed having the balloon opening 134 once the balloon material 1048 is attached as described above.

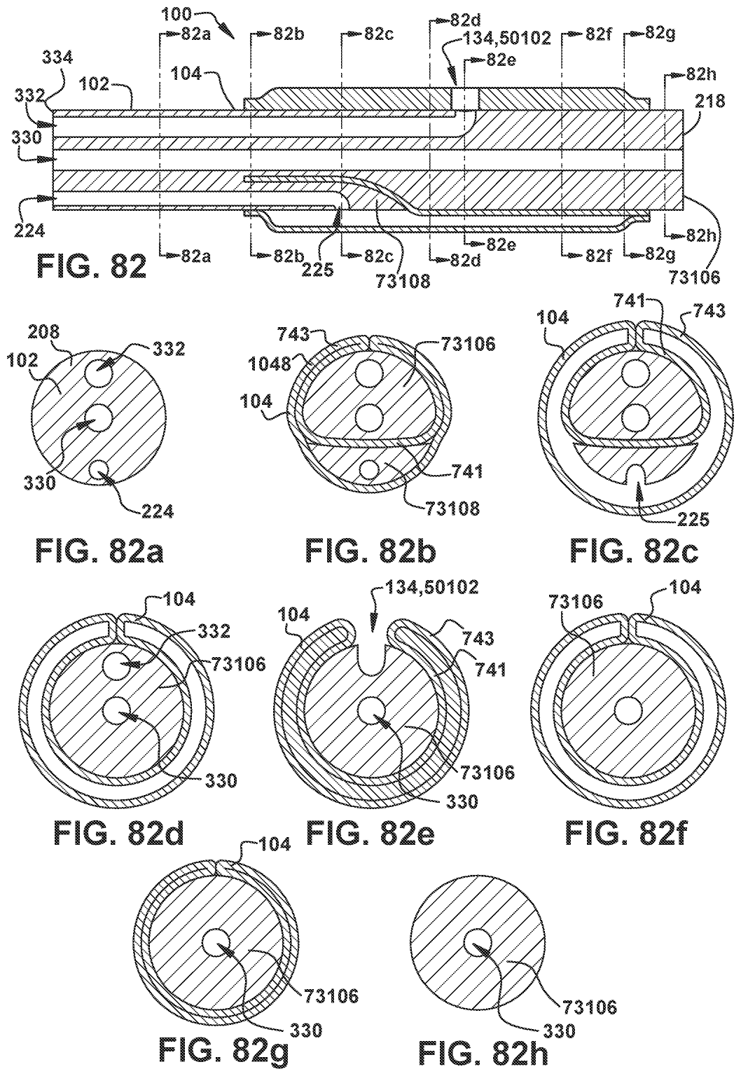

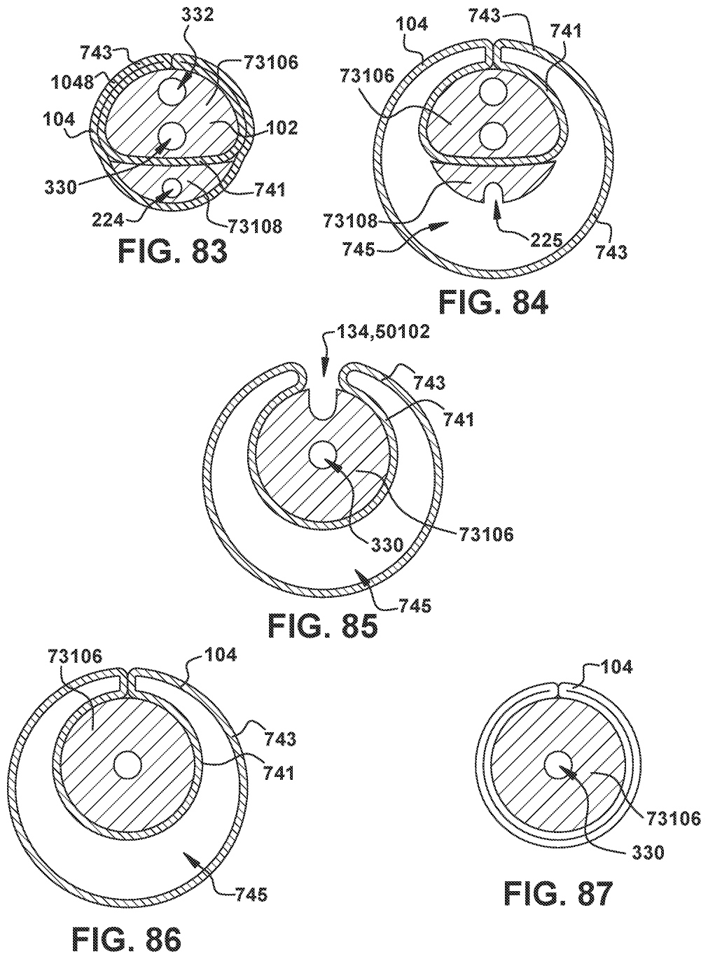

FIG. 82 schematically illustrates the balloon catheter 100 constructed as shown in FIGS. 73-81. As shown in FIG. 82, the first balloon catheter lumen 332 extends from the proximal catheter tip 334, through the primary catheter branch 73106, and to the distal catheter tip 218. The second balloon catheter lumen 332 extends from the proximal catheter tip 334, through the primary catheter branch 73106, and to the balloon opening 134. The inflation opening 224 extends from the proximal catheter tip 334, through the secondary catheter branch 73108, and to the catheter inflation opening 225. FIGS. 82a-h depict cross-sectional views taken at various locations along the balloon catheter 100 of FIG. 82, to show the arrangement of the catheter 102 and the uninflated balloon 104. FIGS. 83-87 depict cross-sectional views of FIGS. 82b, 82c, 82e, 82f, and 82g, respectively, after the balloon 104 has been at least partially inflated. The balloon 104 may be configured such that only the outer balloon portion 743 moves during inflation of the balloon 104 because the inner balloon portion 741 is fixed to the catheter 102 (such as to, e.g., at least the primary and/or the secondary catheter branches 73106, 73108).

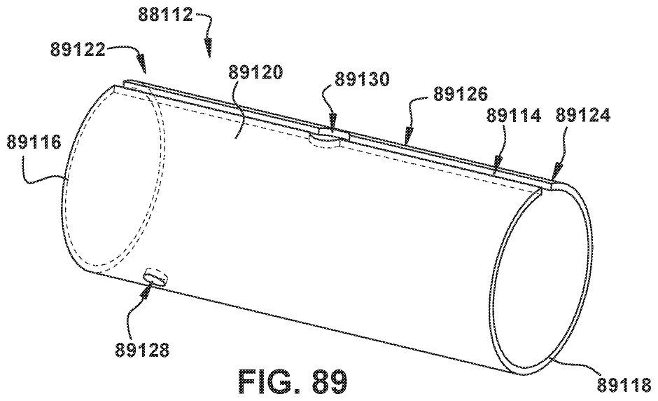

FIG. 88 depicts another example configuration of the balloon catheter 100. The balloon catheter 100 is substantially similar to the balloon catheter 100 of FIG. 69, with some differences. For example, as shown in FIGS. 88 and 88a-f, the balloon catheter 100 includes an outer fixer 88112 between the outer balloon portion 743 and at least one of the inner balloon portion 741 and the catheter 102 (e.g., the outer catheter surface 216). As shown in FIG. 89, the outer fixer 88112 includes an open fixer slit 89114 extending longitudinally between a proximal and distal outer fixer tips 89116, 89118, and radially through an outer fixer sidewall 89120. A proximal outer fixer end portion 89122 includes the proximal outer fixer tip 89116, while an opposite distal outer fixer end portion 89124 includes the distal outer fixer tip 89118. An outer fixer center portion 89126 extends from the proximal outer fixer end portion 89122 to the distal outer fixer end portion 89124.

An outer fixer inflation opening 89128 also extends radially through the outer fixer sidewall 89120. The outer fixer inflation opening 89128 is spaced longitudinally from each of the proximal and distal outer fixer tips 89116, 89118. For example, the outer fixer inflation opening 89128 may be on the outer fixer center portion 89126. As shown in FIGS. 88 and 88a-f, when the balloon catheter 100 is assembled, the outer fixer inflation opening 89128 is aligned and in fluid communication with the catheter inflation opening 225 and the balloon inflation opening 60104 such that the balloon inflation opening 60104 is radially (e.g., directly radially) between the catheter inflation opening 225 and the outer fixer inflation opening 89128. The outer fixer inflation opening 89128 thus fluidically connects the balloon inflation opening 60104 with the inflatable volume 745 of the balloon 104. Further, when assembled, the open fixer slit 89114 is aligned with the balloon opening 134 and the distal catheter sidewall opening 50102. The open fixer slit 89114 may include an enlarged portion 89130 (see, e.g., FIG. 89) that defines a more rounded opening for aligning with both the balloon opening 134 and the distal catheter sidewall opening 50102. FIGS. 88a-f depict cross-sectional views taken at various locations along the assembled balloon catheter 100, to show the arrangement of the catheter 102, the balloon 104, and the outer fixer 88112.

An example sequence of construction for the balloon catheter 100 of FIG. 88 is substantially the same to that shown in FIGS. 60-69, with some differences being described below. For example, after the inner balloon portion 741 at the proximal and distal balloon material ends 1658, 1660 is circumferentially attached to the outer catheter surface 216 via the adhesive 1250 (as is shown and described in relation to FIG. 67), the outer fixer 88112 is inserted over the outer catheter surface 216 and the inner balloon portion 741 such that the outer fixer 88112 is interposed between (e.g., radially between) the inner balloon portion 741 and the outer balloon portion 743. A portion of the balloon material 1048 thus extends through the open fixer slit 89114. The outer fixer 88112 is aligned such that the outer fixer inflation opening 89128 is aligned and in fluid communication with the catheter inflation opening 225 and the balloon inflation opening 60104. Furthermore, the outer fixer 88112 is aligned such that the open fixer slit 89114 is aligned with the balloon opening 134 and the distal catheter sidewall opening 50102. When the open fixer slit 89114 includes the enlarged portion 89130, the enlarged portion 89130 may be aligned with both the balloon opening 134 and the distal catheter sidewall opening 50102.

Once aligned, the outer fixer 88112 is attached to each of the inner balloon portion 741 and the outer catheter surface 216 via an adhesive 1250. For example, the outer fixer center portion 89126 may be directly attached to the inner balloon portion 741, while the proximal and distal outer fixer end portions 89122, 89124 may be directly attached to the outer catheter surface 216. In such case, the inner balloon portion 741 may be directly radially between the outer catheter surface 216 and only the outer fixer center portion 89126. In other words, the proximal and distal outer fixer end portions 89122, 89124 may extend beyond the inner balloon portion 741 so that the proximal and distal outer fixer end portions 89122, 89124 are directly radially adjacent and attached to the outer catheter surface 216, while the outer fixer center portion 89126 is directly radially adjacent and attached to the inner balloon portion 741. Because of this attachment and the provision of the outer fixer 88112, the inner balloon portion 741 thus is “sandwiched” between the outer catheter surface 216 and the outer fixer 88112. The outer fixer 88112 thus helps reinforce and retain the inner balloon portion 741 attached to the outer catheter surface 216.

Once the outer fixer 88112 is attached to the inner balloon portion 741 and the outer catheter surface 216 as described above, the construction of the balloon catheter 100 of FIG. 88 carries on in a similar manner as described in relation to FIGS. 60-69. In particular, the outer balloon portion 743 at the proximal and distal balloon material ends 1658, 1660 is circumferentially attached to at least a portion of at least one of the outer fixer 88112, the inner balloon portion 741, and the outer catheter surface 216 via an adhesive 1250 to form the inflatable volume 745.

Although the outer fixer 88112 has only been shown in the example balloon catheter configuration of FIG. 88, the outer fixer 88112, or a modified version thereof, may be used in any other configuration of the balloon catheter 100 that includes an inner balloon portion 741 fixed to the outer catheter surface 216 and an outer balloon portion 743 that moves relative to the inner balloon portion 741 as the balloon 104 inflates. For example, the outer fixer 88112 may be provided in the balloon catheter 100 of FIGS. 56-57. In such case, the balloon catheter 100 of FIGS. 56-57 with the outer fixer 88112 may be assembled in a similar manner as described above. However, one or more portions of the outer fixer center portion 89126 may be directly radially adjacent and attached to the outer catheter surface 216, while one or more portions of the outer fixer center portion 89126 may be directly radially adjacent and attached to the inner balloon portion 741. Furthermore, the outer fixer inflation opening 89128 may only be aligned with the catheter inflation opening 225 (instead of both a catheter inflation opening 225 and a balloon inflation opening 60104) since the balloon catheter 100 of FIGS. 56-57 does not include a balloon inflation opening 60104.

As another example, the outer fixer 88112 may be provided in the balloon catheter 100 of FIGS. 81-82. In such case, the balloon catheter 100 of FIGS. 81-82 with the outer fixer 88112 may be assembled in a similar manner as described above. A portion of the outer fixer 88112 may be positioned in the space 74110 between the primary catheter branch 73106 and the secondary catheter branch 73108. Furthermore, the outer fixer 88112, when used in conjunction with the balloon catheter 100 of FIGS. 81-82, may have its outer fixer inflation opening 89128 omitted or otherwise altered in order to harmonize with the geometry of the catheter 102.

The balloon catheter 100 is not limited to the example balloon 104 configurations described above and shown in the Figures. For example, the balloon catheter 100 may include, or may include a modified version of, any balloon configuration shown or described in U.S. Pat. No. 11,020,573, granted 1 Jun. 2021 to Karuppasamy and titled “METHOD AND APPARATUSES FOR ACCESSING AND/OR MODIFYING A TARGET PATIENT TISSUE SITE”, the subject matter of which is incorporated by reference in its entirety.

Any example configuration of the balloon catheter 100 and/or the balloon catheter's individual features, when provided, may be at least partially formed from silicone, polyethylene, polypropylene, stainless steel, titanium, rubber, latex, polychloroprene, nylon, any other biocompatible material, or any combination thereof.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages may be obtained from a study of the drawings, the disclosure, and the appended claims.

I claim:

1. A balloon catheter, comprising:
a catheter having longitudinally opposite proximal and distal catheter end portions, the catheter having a catheter inflation opening;
an expandable balloon positioned on at least the distal catheter end portion, the balloon defining at least one balloon opening;
a first balloon catheter lumen extending substantially longitudinally from the proximal catheter end portion toward the distal catheter end portion;
a second balloon catheter lumen extending substantially longitudinally from the proximal catheter end portion to at least the at least one balloon opening; and
an inflation lumen extending substantially longitudinally from the proximal catheter end portion to the catheter inflation opening, the catheter inflation opening providing fluid communication between the inflation lumen and an inflatable volume of the expandable balloon.

2. The balloon catheter of claim 1, wherein the distal catheter end portion has a distal catheter tip, the catheter having an open catheter slit extending from the distal catheter tip toward the proximal catheter end portion, the open catheter slit being aligned with the at least one balloon opening, the balloon catheter further comprising:
a fixer at least partially attaching the expandable balloon to an inner catheter surface of the catheter, the fixer having longitudinally opposite proximal and distal fixer tips and at least one fixer sidewall opening spaced longitudinally from each of the proximal and distal fixer tips, the fixer being disposed within the catheter such that the fixer is at least partially surrounded by the inner catheter surface and the at least one fixer sidewall opening is aligned with both the open catheter slit and the at least one balloon opening, the second balloon catheter lumen extending through the at least one fixer side wall opening.

3. The balloon catheter of claim 2, wherein the first balloon catheter lumen extends from a proximal catheter tip to a distal fixer tip of the fixer, and the second balloon catheter lumen extends from the proximal catheter tip to at least the fixer sidewall opening.

4. The balloon catheter of claim 2, wherein the first balloon catheter lumen extends from a first catheter sidewall opening to a distal fixer tip of the fixer, and the second balloon catheter lumen extends from a second catheter sidewall opening to at least the fixer sidewall opening.

5. The balloon catheter of claim 2, wherein the fixer includes a fixer branch extending from a fixer sidewall adjacent the at least one fixer sidewall opening, the fixer branch having an open branch tip and a branch lumen extending from the open branch tip to the at least one fixer sidewall opening, the fixer branch extending radially into at least one of the open catheter slit and the at least one balloon opening.

6. The balloon catheter of claim 2, wherein the fixer includes at least one fixer blade extending from a fixer sidewall, the at least one fixer blade extending radially into the open catheter slit and between adjacent circumferential end portions of the balloon.

7. The balloon catheter of claim 1, wherein the at least one balloon opening of the balloon includes a plurality of balloon openings, the second balloon catheter lumen extending through each of the balloon openings.

8. The balloon catheter of claim 1, wherein the balloon has a plurality of thin material portions surrounding the at least one balloon opening.

9. The balloon catheter of claim 1, further comprising a wrap encapsulating the balloon, the wrap having at least one wrap opening extending radially therethrough, the at least one wrap opening being aligned with the at least one balloon opening, the second balloon catheter lumen extending from the proximal catheter tip to the at least one wrap opening.

10. The balloon catheter of claim 9, wherein the balloon has a plurality of thin material portions surrounding the at least one balloon opening, the at least one wrap opening being larger than the at least one balloon opening so that the thin material portions extend through the at least one wrap opening when the balloon is inflated.

11. The balloon catheter of claim 9, further comprising a therapeutic substance that is held between the wrap and the balloon, embedded in the wall of the wrap, or embedded in an outer surface of the balloon, inflation of the balloon causing delivery of the therapeutic substance to a target site directly or through a plurality of perforations in the wrap.

12. The balloon catheter of claim 1, wherein the distal catheter end portion has a distal catheter sidewall opening, the at least one balloon opening being aligned with the distal catheter sidewall opening, the first balloon catheter lumen extending substantially longitudinally from the proximal catheter end portion to the distal catheter tip, the second balloon catheter lumen extending substantially longitudinally from the proximal catheter end portion, through the distal catheter sidewall opening and to at least the at least one balloon opening.

13. The balloon catheter of claim 12, wherein the balloon has a balloon inflation opening in fluid communication with an inflatable volume of the balloon, the balloon inflation opening being aligned and in fluid communication with the catheter inflation opening, the inflation lumen extending substantially longitudinally from the proximal catheter end portion to the balloon inflation opening such that inflation fluid is selectably directed into the inflatable volume of the balloon through both the catheter inflation opening and the balloon inflation opening.

14. The balloon catheter of claim 1, wherein
the distal catheter end portion includes a primary catheter branch and a secondary catheter branch each extending longitudinally from the proximal catheter end portion, the primary catheter branch having a distal catheter sidewall opening, the secondary catheter branch having the catheter inflation opening;
the at least one balloon opening is aligned and in fluid communication with the distal catheter sidewall opening;
the first balloon catheter lumen extends substantially longitudinally from the proximal catheter end portion to a distal catheter tip on the primary catheter branch end portion; and
the inflation lumen extends substantially longitudinally from the proximal catheter end portion to the catheter inflation opening in the secondary catheter branch.

15. The balloon catheter of claim 1, wherein the balloon includes inner and outer balloon portions, the inner balloon portion being fixed to at least the catheter such that only the outer balloon portion moves during inflation of the balloon, the inflatable volume of the balloon being between the inner and outer balloon portions.

16. The balloon catheter of claim 15, further comprising an outer fixer having opposing proximal and distal outer fixer tips, a fixer open slit of the outer fixer extending from the proximal outer fixer tip to the distal outer fixer tip, the outer fixer being interposed between the inner and outer balloon portions, the outer fixer being fixed to each of the inner balloon portion and the catheter to reinforce the connection between the inner balloon portion and the catheter, the outer fixer slit being aligned with the at least one balloon opening.

17. The balloon catheter of claim 1, wherein the balloon includes first and second balloon end portions a portion of the first balloon end portion being attached to at least a portion the second balloon end portion in such a manner that the at least balloon opening is defined between the first and second balloon end portions.

18. A method for manipulating a patient lumen, the method comprising:
providing the balloon catheter of claim 1;
inserting a first guidewire into a target patient tissue site in a patient lumen with a first guidewire distal end of the first guidewire being positioned in the patient lumen;
inserting a second guidewire into the target patient tissue site with a second guidewire distal end of the second guidewire being positioned in a patient lumen branch that extends from the patient lumen;
directing a first guidewire proximal end of the first guidewire through the first balloon catheter lumen;
directing a second guidewire proximal end of the second guidewire through the second balloon catheter lumen;
directing the balloon catheter to the target patient tissue site along the first and second guidewires;
positioning the balloon catheter at the target patient tissue site with
at least a portion of the catheter and at least a portion of the expandable balloon being positioned in the patient lumen, and
the balloon opening directed toward the patient lumen branch;
with the balloon catheter positioned at the target patient tissue site, inflating the balloon to dilate the patient lumen;
deflating the balloon after the patient lumen has achieved a predetermined amount of dilation; and
removing the balloon catheter from the target patient tissue site, while at least one of the first and second guidewires are maintained substantially in position at the target patient tissue site.

19. The method of claim 18, wherein as the balloon catheter is directed to the target site, the second guidewire being positioned in the patient lumen branch assists in aligning the balloon opening with an opening of the patient lumen branch by at least partially circumferentially rotating the balloon catheter.

20. The method of claim 18, wherein the balloon has at least one thin material portion at least partially surrounding the at least one balloon opening, and wherein as the balloon expands, the at least one thin material portion expands along the second guidewire into the patient lumen branch.

21. The method of claim 18, wherein the balloon catheter has an expandable implant mounted thereon, the method further including:
with the balloon catheter at the target patient tissue site, expanding the expandable implant at least partially via the inflating of the balloon; and
with the expandable implant expanded, deflating the balloon; and
with the balloon deflated, removing the balloon catheter from the target patient tissue site, while at least one of the first guidewire, the second guidewire, and the expandable implant are maintained substantially in position at the target patient tissue site.
